(19) United States
(12) Patent Application Publication
(10) Pub. No.: US 2013/0256227 A1
(43) Pub. Date: Oct. 3, 2013

(54) POLYSTYRENE SULFONATE RESIN FOR USE WITH A HEMODIALYSIS SYSTEM HAVING A CONTROLLED COMPLIANCE DIALYSIS CIRCUIT

(71) Applicants: Thomas D. Kelly, Highland Park, IL (US); SuPing Lyu, Maple Grove, MN (US)

(72) Inventors: Thomas D. Kelly, Highland Park, IL (US); SuPing Lyu, Maple Grove, MN (US)

(73) Assignee: MEDTRONIC, INC., Minneapolis, MN (US)

(21) Appl. No.: 13/612,701

(22) Filed: Sep. 12, 2012

Related U.S. Application Data
(60) Provisional application No. 61/533,775, filed on Sep. 12, 2011.

(51) Int. Cl. A61M 1/36

(52) U.S. Cl. CPC A61M 1/3621
USPC 210/638; 210/483; 210/137; 210/96.2

(57) ABSTRACT

Sorbent cartridges having a polystyrene sulfonate resin saturated with calcium ions for the performance of kidney replacement therapy are disclosed. Systems and methods having or using a sorbent cartridge, a dialyzer, control components, a cartridge having a polystyrene sulfonate resin, and fluid reservoirs configured to be of a weight and size suitable to be worn or carried by an individual requiring treatment are disclosed. A system for performing kidney replacement therapy has a controlled compliance dialysis circuit, where a control pump controls the bi-directional movement of fluid across a dialysis membrane. The system provides for the monitoring of an inlet and outlet conductivity of the sorbent cartridge to quantify or monitor the removal of urea by the sorbent cartridge.

### CROSS REFERENCE TO RELATED APPLICATIONS

This application claims the benefit of priority to U.S. Provisional Patent Application Ser. No. 61/533,775, filed on Sep. 12, 2011, which is incorporated by reference.

### FIELD OF THE INVENTION

The invention relates to an apparatus for hemodialysis and hemofiltration for the treatment of pathological conditions such as End Stage Renal Disease (ESRD) on a frequent or continuous basis. The systems and methods include a system having a dialyzer, control components, dialysate regeneration cartridge and fluid reservoirs configured to be of a weight and size to be worn or carried by an individual requiring treatment. Further, the systems and methods include a polystyrene sulfonate resin cartridge for the precipitation of specific anions from a dialysate. The disclosure further relates to the treatment of Chronic Kidney Disease (CKD) through methods and apparatuses that allow an individual to remain ambulatory during treatment.

### BACKGROUND

Kidney failure affects the kidneys' ability to clear waste products from the blood of an individual. The most acute symptom of kidney failure is the build-up of urea in the blood, where urea is the product of nitrogen metabolism in the body. However, the kidneys are also involved in the homeostasis of other blood components including calcium, phosphates and sulfate. In particular, kidney failure reduces the ability to clear phosphates from the blood and results in an increase in parathyroid hormone levels. The skeletal system is the major site of storage of both calcium and phosphates in the body, where a disruption in phosphate homeostasis also has a tendency to affect calcium levels in the blood. Patients receiving kidney dialysis frequently take phosphate binder drugs to prevent the absorption of phosphates from the diet. Inorganic sulfate is a physiologic anion necessary for certain detoxification and biosynthetic reactions whose levels are under tight homeostasis control. Removal of these anions from the blood by the kidneys is clearance limited, which can result in elevated anion levels in kidney failure patients. Existing dialysis sorbents are not particularly effective in removing sulfates. Koopman et al. reported that sulfate accumulates to a high level under sorbent dialysis, whereas during single pass dialysis sulfate is efficiently removed (Koopman B J, Jansen G, Wolthers B G, Beukhof J R, Go J G; Determination of inorganic sulfate in plasma of patient receiving different types of haemodialysis. J Chromatography 1985 Feb. 8; 337 (2): 259-66).

During operation of typical dialysis machines to perform hemodialysis, blood is passed through a dialysis chamber on one side of a dialysis membrane and a dialysate is passed on the other side of the dialysis membrane. In addition to diffusion of solutes across the dialysis membrane, a difference in pressure between the blood-side and the dialysate-side of the dialysis membrane drives the bulk movement of water from higher pressure to lower pressure. The pressure generated on a particular side of the dialysis membrane depends on several factors including flow rate, viscosity of the fluid, geometry of the dialyzer and physiological condition of the patient. The diffusion of impurities from the blood, across the dialysis membrane and into the dialysate is thermodynamically driven by the concentration gradient difference between the concentration of impurities in the blood and the concentration of those species in the dialysate. In certain dialysis systems, the same volume of dialysate is repeatedly used to perform dialysis. However, in order to maintain a concentration gradient between the blood and the dialysate, waste products that diffuse into the dialysate must be continually removed along with any impurities derived from external sources.

Prior portable dialysis systems tend to rely upon the regeneration of spent dialysate (i.e. dialysate having urea and/or other impurities therein) to form refreshed dialysate that can be reused to perform dialysis. Regeneration of the dialysate allows for the volume of fluid that needs to be supplied to perform a session of dialysis treatment to be limited and enable a portable system. Some systems employ a reservoir of working dialysis solution that varies in volume depending upon bulk movement of water across the dialysis membrane and/or water added to dilute sodium ion concentration and reduce conductivity. However, systems where the volume of working dialysate varies during the course of treatment complicate accurate control over removal of fluid from a patient through techniques such as ultrafiltration and diafiltration.

The large volume of dialysate fluid required for dialysis is in part attributable to the large quantity of water necessary for the dissolution of waste products removed from the blood of a dialysis patient, and the electrolytes within the dialysate. Regeneration of spent dialysate is one method to reduce the total volume of a dialysis system by eliminating the need for a large reserve of fresh dialysate. In order for spent dialysate to be reused, accumulated waste products and impurities must be removed from the spent dialysate, and the composition and pH of the regenerated dialysate must be regulated for physiological compatibility. Devices that regenerate spent dialysis fluid for reuse are primarily directed toward the removal of urea, ammonium ions, uric acid, creatinine, and phosphate via various sorbents. For example, the Recirculating Dialysate System (“REDY system”), which was introduced in the 1970s, employs a sorbent cartridge through which spent dialysate is recirculated and regenerated. However, the regenerated dialysate produced by REDY systems is subject to variations in pH and sodium concentrations non-conducive to physiological norms. Additionally, REDY systems have limited or no ability to remove sulfates.

Moreover, traditional dialysis systems employing sorbent technology, such as the REDY system usually employ low-flux dialyzers and adjust dialysate pressure to achieve net patient fluid removal. The UF coefficient of a dialyzer specifies the rate of filtration through the dialyzer due to pressure differences across the dialyzer membrane, typically called the trans-membrane pressure. The trans-membrane pressure is calculated by the formula TMP=((Blood Inlet Pressure+Blood Outlet Pressure)/2)−((Dialysate Inlet Pressure+Dialysate Outlet Pressure)/2). This formula is usually shortened to TMP=Venous Return Pressure−Dialysate Pressure. Low-flux hemodialyzers have a UF coefficient of less than 8 ml of water flux per hour per mmHg of trans-membrane pressure. To illustrate fluid removal with the traditional sorbent system, a typical low flux dialyzer could have a UF coefficient of 4 mL/hr/mmHg. To calculate the pressure necessary to achieve the rate of fluid removal, the desired hourly fluid removal is divided by the dialyzer UF coefficient. For example, an hourly rate of 0.5 L/hr yields a required trans-membrane pressure (TMP) of 125 mmHg if the UF coefficient is 4 mL/hr/mmHg. 125 mmHg is the trans-membrane pressure required to remove fluid at a rate of 0.5 L per hour. The venous pressure is a function of the blood flow rate and the blood return restriction (needle and access). As the Venous Return Pressure cannot be set, to control the fluid removal rate it is necessary to calculate the required dialysate pressure. The operator calculates dialysate pressure via the formula Dialysate Pressure=Venous Pressure−TMP, if the venous return pressure were 75 mmHg, (DP=75−125=−50 mmHg). In this example the user must adjust the dialysate pressure to −50 mmHg to achieve the TMP of 125 mmHg. The venous pressure fluctuates during treatment so the operator must adjust the dialysate pressure on a regular basis, which is not suitable for a non-medical professional or a home patient. With high-flux dialyzers, pressure alone is not accurate enough to control ultrafiltration because fluid moves more freely across the dialyzer membrane. To control ultrafiltration in conventional hemodialysis using high-flux dialyzers, balancing chambers, flow sensors or other methods to balance flow to and from the dialyzer are employed. In CRRT (continuous renal replacement therapy) equipment, pumps controlled by precise scales are required to control the flow to and from the dialyzer very accurately.

Subsequent development of dialysate recirculating techniques has resulted in systems that employ a variety of sorbent media, including activated carbon, urease, and zirconium-, aluminum-, and magnesium-based materials. One problem associated with sorbent regeneration of spent dialysate is the buildup of sodium ions released as a byproduct of the adsorption process, thus necessitating a high degree of sodium concentration control which has yet to be achieved by current wearable or portable dialysis systems. Deionization resins have been explored to combat the buildup of sodium ions with mixed results. Electrolytes such as calcium, magnesium, and potassium are removed from spent dialysate by sorbent and deionization media and must be added back to the dialysate prior to reuse. Additionally, carbon dioxide gas is generated during the absorption process, especially in systems employing urease, and accumulates in the dialysate. Accordingly, sorbent-based dialysis regeneration systems typically must maintain large reservoirs of sterile water and electrolytic solutions to regulate sodium concentration and maintain electrolyte concentration, respectively, and include a means for removing accumulated carbon dioxide gas, thus defeating the intended purpose of reducing total system volume and size.

A typical sorbent cartridge passes dialysate entering the cartridge through the urease-containing material, the zirconium phosphate material, the zirconium oxide material and the activated carbon material. Some sorbent cartridges can contain an additional activated carbon material that contacts the used dialysate prior to the urease-containing material to remove metal ions or fluorine ions that can damage the urease-containing material. Similarly, a single activated carbon material can also be placed prior to the urease-containing material such that the activated carbon material is the first material contacted by dialysate entering the column. In other versions, uremic toxin-treating enzyme particles are intermixed with cation exchange particles as described in WO 2009/157877. However, zirconium-based exchange materials are particularly expensive due to the high cost of zirconium. Further, the zirconium-based exchange materials release ions into the dialysate that affect conductivity and/or the pH of the dialysate, which necessitates the addition of further reagents to the dialysate to maintain the composition of the dialysate. For example, the zirconium oxide component absorbs phosphates; however, acetate and bicarbonate are released at the same time. Acetate in the dialysis circuit can lead patients to experience acetate intolerance. Similarly, the zirconium phosphate material releases a significant amount of sodium ions in exchange for ammonium ions. Over the course of treatment, the concentration of sodium ions can increase the overall conductivity of the dialysate to an unsafe level.

Some systems have attempted to address the volume and weight problems by allowing for external connections to a tap water source in order to replenish system volume as needed. However, the introduction of tap water into a dialysis system necessitates additional purification measures, thus adding to system complexity and size. As a result, such systems are sometimes not useful for mobile or portable use.

U.S. Pat. No. 3,669,878 Marantz et al. describes sorbent removal of urea and ammonium ions from spent dialysate via urease, ammonium carbonate, and zirconium phosphate, U.S. Pat. No. 3,669,880 Marantz et al. describes directing a controlled volume of dialysate through zirconium phosphate, activated carbon, and hydrated zirconium oxide columns, U.S. Pat. No. 3,850,835 Marantz et al. describes production of a zirconium hydrous oxide ion exchange media, and U.S. Pat. No. 3,989,622 Marantz et al. describes adsorption of urease on aluminum oxide and magnesium silicate media to convert liquid urea to ammonium carbonate. Additional information can be found in U.S. Pat. No. 4,581,141 Ash, which describes removal of uremic substances from dialysate via a calcium-based cation exchanger, urease, and aliphatic carboxylic acid resin. U.S. Pat. No. 4,826,663 Alberti et al. describes a method of preparing a zirconium phosphate ion exchanger. U.S. Pat. No. 6,627,164 Wong describes production of sodium zirconium carbonate for ion exchange in renal dialysis, and U.S. Pat. No. 7,566,432 Wong describes production of zirconium phosphate particles for ion exchange in regenerative dialysis. U.S. Pat. No. 6,818,196 Wong, U.S. Pat. No. 7,736,507 Wong, U.S. Application Publication 2002/0112609 Wong, U.S. Application Publication 2010/0078387 Wong, and U.S. Application Publication 2010/00784330 Wong, describe cartridges for purification of dialysis solutions using sodium zirconium carbonate.

U.S. Pat. No. 6,878,283 Thompson, U.S. Pat. No. 7,776,210 Rosenbaum et al., U.S. Application Publication 2010/0326911 Rosenbaum et al., U.S. Application Publication 2010/0078381 Merchant, U.S. Application Publication 2009/0127193 Updyke et al. and U.S. Application Publication 2011/0017665 Updyke et al. describe filter cartridges having a plurality of types of filter media including zirconium compounds, urease, and alumina for dialysis systems. U.S. Pat. No. 4,460,555 Thompson and U.S. Pat. No. 4,650,587 Polak et al. describe magnesium phosphate media for removal of ammonia from aqueous solutions. U.S. Application Publication 2009/0282980 Gura et al. describes degassing devices for use in dialysate systems having urease media. WO 2009/157877 A1 describes a urease material having urease immobilized on a substrate intermixed with a cation exchange material or zirconium phosphate material to improve workability for the reduction of clogging and to improve absorption of ammonium ions generated by the urease.

There is a need for a mobile dialysis system having a weight and volume of the system that is sufficiently small in order to be comfortably born by the patient while ambulatory. Further, to facilitate regular usage, the system should be conducive to operation by a patient, without the assistance of a medical professional. Accordingly, there remains a need for a patient-friendly wearable and/or portable dialysis system that is capable of operating on a small volume of dialysate and suitable for daily continuous or short-term dialysis.

### SUMMARY OF THE INVENTION

The invention is directed toward a system for kidney replacement therapy having a polystyrene sulfonate resin cartridge for the removal of impurities and waste species for a dialysate.

In any embodiment, the polystyrene sulfonate resin cartridge contains a polystyrene sulfonate resin saturated with calcium ions capable of removing one or more of carbonate ions, sulfate ions and phosphates from a dialysate.

In any embodiment, a polystyrene sulfonate resin removes one or more of sodium ions, carbonate ions, sulfate ions and phosphates from a fluid passing through the cartridge.

In any embodiment, a polystyrene sulfonate resin is substantially saturated with calcium ions.

In any embodiment, a polystyrene sulfonate resin saturated with calcium ions can be substantially saturated with calcium ions.

In any embodiment, the polystyrene sulfonate resin predominately contains the following structure:

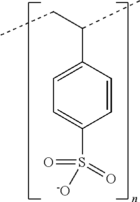

where n is an integer from about 5 to about 200.

In any embodiment, a polystyrene sulfonate resin saturated with calcium ions ((RSO₃⁻)₂Ca²⁺) precipitates carbonate and absorbs sodium ions through the following reactions:

(RSO₃⁻)₂Ca²⁺+Na⁺ [symbol] 2RSO₃⁻Na⁺+Ca²⁺ (1)

Ca²⁺(aq)+CO₃²⁻(aq)→CaCO₃ (2).

In any embodiment, a polystyrene sulfonate resin saturated with calcium ions ((RSO₃⁻)₂Ca²⁺) precipitates sulfate and absorbs sodium ions through the following reactions:

(RSO₃⁻)₂Ca²⁺+Na⁺ [symbol] 2RSO₃⁻Na⁺+Ca²⁺ (1)

Ca²⁺(aq)+SO₄²⁻(aq)→CaSO₄ (3).

In any embodiment, a polystyrene sulfonate resin saturated with calcium ions ((RSO₃⁻)₂Ca²⁺) precipitates HₓPO₄^{3-x} species and absorbs sodium ions through the following reactions:

(RSO₃⁻)₂Ca²⁺+Na⁺ [symbol] 2RSO₃⁻Na⁺+Ca²⁺ (1)

Ca²⁺(aq)+HₓPO₄^{x-3}(aq)→Ca₃(PO₄)₂(s)+xH⁺ (4),

where x is an integer from 0 to 2.

In any embodiment, a polystyrene sulfonate resin cartridge can be used in any hemodialysis, ultrafiltration, or hemodiafiltration system.

The invention is further directed to a system for kidney replacement therapy having a size and weight suitable to be carried or worn by a patient during a dialysis treatment. In any embodiment, a method for performing replacement kidney therapy is provided having the steps of attaching the vasculature of a subject to a system for kidney replacement therapy having an extracorporeal circuit having a first end that draws blood from the subject and a second end that returns blood to the subject, and a dialysis circuit. The extracorporeal circuit and the dialysis circuit are in fluid communication through a dialysis membrane housed in a dialyzer. Blood from the subject is conveyed via a blood pump through the extracorporeal circuit and the dialyzer and returned to the patient. A dialysate is conveyed through a dialysis circuit via a dialysate pump such that the dialysate moves from a sorbent cartridge, to the dialyzer and back to the sorbent cartridge, where at least one waste species diffuses from the blood to the dialysate through the dialysis membrane and the sorbent cartridge substantially removes at least one impurity or waste species from the dialysate, and wherein the dialysate is intermittently conveyed through a polystyrene sulfonate resin cartridge containing a polystyrene sulfonate resin saturated with calcium ions (Ca²⁺) and the polystyrene sulfonate resin cartridge removes at least one impurity of waste species from the dialysate.

In any embodiment, the impurity or waste species removed by the polystyrene sulfonate resin cartridge is selected from the group consisting of carbonate ions, sulfate ions and phosphates.

In any embodiment, a system is provided for performing kidney replacement therapy having a dialysis membrane, a blood inlet end for receiving blood, a blood outlet end for allowing blood out of the dialyzer, a dialysate inlet end for receiving dialysate and a dialysate outlet end for allowing dialysate out of the dialyzer, wherein the blood and the dialysate contact different sides of the dialysis membrane. The system has an extracorporeal circuit having a conduit for receiving blood from a subject and a conduit for returning blood to the subject, a blood pump for conveying blood from the subject through the extracorporeal circuit and the dialyzer, wherein blood is conveyed from the subject, to the dialyzer and back to the subject. The system has a dialysis circuit having a sorbent cartridge for removing at least one waste species from the dialysate, one or more conduits for carrying dialysate between the sorbent cartridge and the dialyzer, and a dialysate pump for conveying dialysate from the sorbent cartridge, to the dialyzer and back to the sorbent cartridge, and a polystyrene sulfonate resin cartridge and one or more conduits for carrying dialysate from the dialysis circuit through the polystyrene sulfonate resin cartridge. The sorbent cartridge has a dialysate inlet end and a dialysate outlet end and the polystyrene sulfonate resin cartridge contains a polystyrene sulfonate resin saturated with calcium ions, where the polystyrene sulfonate resin cartridge removes one or more selected from the group consisting of carbonate ions, sulfate ions and phosphates from the dialysate.

In any embodiment, the dialysate being conveyed from the dialyzer to the sorbent cartridge can be intermittently conveyed through the polystyrene sulfonate resin cartridge or dialysate being conveyed from the sorbent cartridge to the dialyzer can be intermittently conveyed through the polystyrene sulfonate resin cartridge.

In any embodiment, the polystyrene sulfonate resin cartridge is provided having a polystyrene sulfonate resin saturated with calcium ions (Ca²⁺) and capable of removing one or more selected from the group consisting of carbonate ions, sulfate ions and phosphates.

In any embodiment, the system and the polystyrene sulfonate resin cartridge is used in any one of hemodialysis, ultrafiltration, or hemodiafiltration.

In any embodiment, a system for performing kidney replacement has an extracorporeal circuit and a dialysis circuit, wherein the dialysis circuit has a sorbent cartridge for removing at least one waste species from a dialysate, one or more conduits for carrying dialysate between the sorbent cartridge and a dialyzer, and a dialysate pump for conveying dialysate from the sorbent cartridge, to the dialyzer and back to the sorbent cartridge. A polystyrene sulfonate resin cartridge and one or more conduits are provided for carrying dialysate from the dialysis circuit and through the polystyrene sulfonate resin cartridge, wherein the polystyrene sulfonate resin cartridge contains a polystyrene sulfonate resin saturated or substantially saturated with calcium ions and the polystyrene sulfonate resin cartridge removes one or more selected from the group consisting of sodium, carbonate ions, sulfate ions and phosphates from the dialysate.

In any embodiment, a portion of a dialysate being conveyed from the sorbent cartridge to the dialyzer passes through the polystyrene sulfonate resin cartridge.

In any embodiment, a portion of the dialysate being conveyed from the dialyzer to the sorbent cartridge passes through the polystyrene sulfonate resin cartridge.

In any embodiment, one or more controllers control a flow of dialysate through a sorbent cartridge and through a polystyrene sulfonate resin cartridge.

In any embodiment, a system has a first conductivity meter for measuring the conductivity of dialysate at a dialysate inlet end of a sorbent cartridge; a second conductivity meter for measuring the conductivity of the dialysate at a dialysate outlet end of the sorbent cartridge; and one or more controllers for comparing the conductivity measured by the first conductivity meter and the second conductivity meter and calculating the amount of urea absorbed by the sorbent cartridge.

In any embodiment, one or more controllers calculate an amount of urea absorbed by a sorbent cartridge.

In any embodiment, one or more controllers display the amount of urea absorbed by the sorbent cartridge to the subject or other individual.

In any embodiment, one or more controllers signals an alert if the conductivity measured by the first conductivity sensor and the second conductivity sensor are substantially equal.

In any embodiment, a system can prevent the conveyance of dialysate to the dialyzer.

In any embodiment, a void volume of the dialysis circuit is substantially filled with a volume of dialysate, wherein the volume of dialysate does not substantially change during hemodialysis treatment.

In any embodiment, a dialysis circuit is a controlled compliance dialysis circuit having a control pump and a control reservoir, wherein the control pump is configured to add fluid to the dialysis circuit or remove fluid from the dialysis circuit.

In any embodiment, a volume of fluid added to a dialysis circuit by a control pump results in substantially the same volume of fluid transferred to the body of a subject.

In any embodiment, a net volume of fluid removed from a dialysis circuit by the control pump results in substantially the same volume of fluid removed from the body of a subject.

In any embodiment, a rate of a blood pump for conveying blood through the extracorporeal circuit and a dialysate pump are controlled such that a ratio of the flow of blood to dialysate through a dialyzer is from about 1:1.5 to about 3:1.

In any embodiment, a control pump is intermittently switched between an efflux direction and an influx direction such that the control pump is not operated in either the efflux direction or the influx direction for a time period exceeding about 2 minutes, wherein operation of the control pump in the efflux direction removes fluid from a subject's body via an extracorporeal circuit and operation of the control pump in the influx direction adds fluid to the subject's body via the extracorporeal circuit.

In any embodiment, a control pump is operated in a manner to cause the net removal of about 100 to about 1000 mL of fluid from the subject's body per hour of treatment.

In any embodiment, a system has a deionization cartridge having a mixed bed deionization resin.

In any embodiment, a system has a hematocrit sensor to determine a hematocrit level of blood in an extracorporeal circuit, wherein the hematocrit sensor is configured to send information to one or more controllers.

In any embodiment, a system has a relative blood volume monitor to determine the relative blood volume hydration status (RBVHS) of blood in an extracorporeal circuit, wherein the relative blood volume monitor is configured to send information to the one or more controllers.

In any embodiment, a relative blood volume monitor determines the level of one or more solutes in the blood at a first time (C₀) and determines the level of the one or more solutes in the blood at a second time later than the first time (Cₜ), and the relative blood volume hydration status is calculated by the formula RBVHS=C₀/Cₜ.

In any embodiment, a relative blood volume monitor is a hematocrit sensor.

In any embodiment, a hematocrit sensor is an oximeter.

In any embodiment, a hematocrit sensor has a light source for emitting red or infrared light and a detector for detecting the emitted light.

In any embodiment, a relative blood volume monitor measures the velocity of ultrasonic sound waves in the blood in an extracorporeal circuit.

In any embodiment, a relative blood volume monitor measures the velocity of ultrasonic sound waves in the blood in an extracorporeal circuit, wherein the velocity of ultrasonic sound waves indicates the level of protein concentration in the blood.

In any embodiment, a system has an impedance detector for determining a tissue fluid volume in a subject, wherein the impedance detector is configured to send information to one or more controllers.

In any embodiment, a relative blood volume monitor is configured to determine the fluid volume of blood at a position prior to the blood entering a dialyzer.

In any embodiment, wherein one or more controllers operates a control pump to maintain a ratio of tissue fluid volume to blood fluid volume in the range from about 6:1 to about 9:1.

In any embodiment, as system has a second control reservoir and a second reservoir pump, wherein the second control reservoir holds a fluid that can be added to the dialysis circuit by operation of the second reservoir pump.

In any embodiment, a second reservoir contains water, tap water or purified water.

In any embodiment, a system for kidney replacement therapy is operated through a method where the vasculature of a subject is attached to a system for kidney replacement therapy having an extracorporeal circuit having a first end that draws blood from the subject and a second end that returns blood to the subject, and a dialysis circuit. The extracorporeal circuit and the dialysis circuit are in fluid communication through a high permeability dialysis membrane housed in a dialyzer. Blood is conveyed from the subject through the extracorporeal circuit and the dialyzer and returned to the subject. A dialysate is conveyed through the dialysis circuit such that the dialysate moves from a sorbent cartridge, to the dialyzer and back to the sorbent cartridge, where at least one waste species diffuses from the blood to the dialysate through the dialysis membrane and the sorbent cartridge substantially removes at least one impurity or waste species from the dialysate. Intermittently, a portion of the dialysate is conveyed through a polystyrene sulfonate resin cartridge, wherein the polystyrene sulfonate resin cartridge contains a polystyrene sulfonate resin, and wherein the polystyrene sulfonate resin cartridge removes one or more selected from carbonate, sulfate and phosphate ions from the dialysate. The dialysate can be conveyed through the polystyrene sulfonate resin cartridge as the dialysate is being conveyed from the dialyzer to the sorbent cartridge or from the sorbent cartridge to the dialyzer. The dialysis circuit can contain a deionization cartridge containing a mixed bed deionization resin.

In any embodiment, a flow of dialysate through the polystyrene sulfonate resin cartridge and a flow of dialysate bypassing the polystyrene sulfonate resin cartridge are controlled based upon the conductivity of the dialysate in the dialysis circuit. Flow of dialysate through the polystyrene sulfonate resin cartridge and a flow of dialysate bypassing the polystyrene sulfonate resin cartridge are controlled based upon the level of impurities from the patient's blood present in the dialysate.

In any embodiment, the dialysate being conveyed from the dialyzer to the sorbent cartridge can be conveyed through the polystyrene sulfonate resin cartridge or dialysate being conveyed from the sorbent cartridge to the dialyzer can be conveyed through the polystyrene sulfonate resin cartridge.

In any embodiment, the system has a control reservoir and a control pump, wherein the control pump can add fluid to the dialysis circuit from the control reservoir or remove fluid from the dialysis circuit to the control reservoir.

In any embodiment, the system has a second control reservoir and a second reservoir pump, wherein the second control reservoir holds a fluid that can be added to the dialysis circuit by operation of the second reservoir pump.

In any embodiment, a flow of dialysate through the polystyrene sulfonate resin cartridge and a flow of dialysate bypassing the polystyrene sulfonate resin cartridge are controlled based upon the concentration of calcium ions in the dialysate.

In any embodiment, a blood hydration status monitor monitors the relative blood hydration status of the subject's blood in the extracorporeal circuit or a hematocrit detector monitors the hematocrit of the subject's blood in the extracorporeal circuit.

In any embodiment, enhanced convective clearance is performed utilizing the controlled compliance dialysis circuit by operating the control pump in a bidirectional manner with intermittent reversal of the direction of operation.

In any embodiment, a system has a sorbent cartridge for regenerating a dialysate and a dialyzer performing hemodialysis and/or ultrafiltration, wherein the dialyzer exchanges water, urea, NaCl, electrolytes, and waste substances between blood and a dialysate during hemodialysis and removes water from blood during ultrafiltration. The dialyzer is incorporated in a housing having a blood inlet and a blood outlet, and a dialysate inlet and a dialysate outlet of the housing in fluid communication with the sorbent cartridge.

In any embodiment, the system has a polystyrene sulfonate resin cartridge and conduits for carrying at least a portion of the dialysate from the dialysis circuit through the polystyrene sulfonate resin cartridge.

In any embodiment, the system has an infusate reservoir containing an infusate containing one or more electrolytes selected from potassium ions, calcium ions, and magnesium ions. The infusate is added to the dialysate under the control of a controller in order to maintain the concentration of potassium ion, calcium ion and/or magnesium ion within predetermined ranges.

In any embodiment, the dialysate is conveyed through the system by a dialysate pump, where the rate of the dialysate pump is controlled by a controller. In any embodiment, blood from a patient is conveyed through the system by a blood pump, wherein the blood pump is controlled by a controller. In any embodiment, the pressure of blood entering the dialyzer forming part of the system is measured entering the dialyzer by a pressure meter, and the pressure of the blood exiting the dialyzer is measured by a pressure meter.

In any embodiment, a system has a use for the performance of kidney replacement therapy. The system has an extracorporeal circuit having a first end that draws blood from a subject and a second end that returns blood to the subject, and a dialysis circuit. The extracorporeal circuit and the dialysis circuit are in fluid communication through a dialysis membrane housed in a dialyzer. The system is further configured to perform a method for kidney replacement therapy of conveying blood from the subject via a blood pump through the extracorporeal circuit and the dialyzer and returning the blood to the subject; conveying a dialysate through the dialysis circuit via a dialysate pump such that the dialysate moves from a sorbent cartridge, to the dialyzer and back to the sorbent cartridge, where at least one waste species diffuses from the blood to the dialysate through the dialysis membrane and the sorbent cartridge substantially removes at least one impurity or waste species from the dialysate; and intermittently conveying the dialysate through a polystyrene sulfonate resin cartridge as the dialysate is being conveyed from the dialyzer to the sorbent cartridge or from the sorbent cartridge to the dialyzer. The polystyrene sulfonate resin cartridge contains a polystyrene sulfonate resin saturated with calcium ions and the polystyrene resin cartridge removes at least one waste or impurity species from the dialysate.

In any embodiment, a method for performing kidney replacement therapy is provided. A fluid or dialysate is conveyed through a dialysis circuit via a dialysate pump such that the pump conveys the fluid or dialysate from a sorbent cartridge, to a dialyzer and back to the sorbent cartridge, wherein at least one impurity or waste species diffuses into the fluid or dialysate via the dialyzer and the sorbent cartridge removes the waste species from the external fluid and releases sodium ions into the fluid or dialysate. The fluid or dialysate is intermittently conveyed through a polystyrene sulfonate resin cartridge, wherein the polystyrene sulfonate resin cartridge contains a polystyrene sulfonate resin saturated or substantially saturated with calcium ions and the polystyrene sulfonate resin cartridge removes one or more selected from sodium ions, carbonate ions, sulfate ions and phosphates from the external fluid.

In any embodiment, an external fluid is intermittently conveyed through the polystyrene sulfonate resin as the fluid or dialysate is being conveyed from the dialyzer to the sorbent cartridge or from the sorbent cartridge to the dialyzer

In any embodiment, a portion of the fluid or dialysate is conveyed through a second bypass pathway of a dialysis circuit such that the external fluid moves from an outlet of the sorbent cartridge to an inlet of the sorbent cartridge without passing through the dialyzer.

In any embodiment, a dialysate or fluid is conveyed through a second bypass pathway based at least in part on the conductivity measured of the dialysate or fluid.

In any embodiment, a dialyzer has a dialysis membrane having high permeability.

In any embodiment, at least one waste species removed by a sorbent cartridge includes urea.

In any embodiment, the conductivity of the dialysate or fluid is monitored at an inlet end of a sorbent cartridge via a first conductivity meter; conductivity of the external fluid at an outlet end of the sorbent cartridge via a second conductivity meter is monitored; and an amount of urea absorbed by the sorbent cartridge is calculated based at least in part upon the conductivity measured at the inlet end of the sorbent cartridge and at the outlet end of the sorbent cartridge.

In any embodiment, an amount of urea absorbed by the sorbent cartridge is calculated by calculating a starting conductivity by subtracting a conductivity attributed to Ca²⁺, Mg²⁺, and K⁺ ions in the external fluid from a conductivity measured at the inlet of the sorbent cartridge; calculating a corrected outlet conductivity by subtracting an increase in conductivity attributed to an exchange of Ca²⁺, Mg²⁺, and K⁺ ions for Na⁺ ions by the sorbent cartridge from a conductivity measured at the outlet of the sorbent cartridge; and calculating a conductivity increase from the exchange of NH₄⁺ for Na⁺ ions by the sorbent cartridge by subtracting the starting conductivity from the corrected outlet conductivity.

In any embodiment, an alert is signaled if the conductivity measured by a first conductivity meter and a second conductivity meter is substantially equal.

In any embodiment, an alert is signaled if the conductivity measured by a first conductivity meter and a second conductivity meter is substantially equal and conveyance of the dialysate or fluid to the dialyzer is stopped.

In any embodiment, a pump for conveying a fluid, dialysate or blood is a peristaltic pump.

In any embodiment, a pump for conveying a fluid, dialysate or blood is not a pulsatile pump.

In any embodiment, a dialysis circuit has an infusate container containing an infusate solution and an infusate pump for adding the infusate solution to the circuit, the infusate solution comprising a potassium salt.

In any embodiment, an infusate solution does not comprise a calcium salt.

In any embodiment, an infusate solution has a magnesium salt.

In any embodiment, a pump for conveying a fluid, dialysate or blood operates at a rate from about 10 to about 400 mL/min.

In any embodiment, a total volume of a dialysate or fluid within the circuit is less than about 1 L.

In any embodiment, a dialysis circuit has an air trap for removing air from the fluid, the air trap located between an outlet end of a sorbent cartridge and a dialyzer.

In any embodiment, a dialysis circuit is a controlled compliance circuit.

In any embodiment, a ratio of dialysate or fluid flow passing through the polystyrene sulfonate resin cartridge and dialysate or fluid flow passing through only the sorbent cartridge is controlled, wherein the external fluid flow passing through the polystyrene sulfate resin cartridge is represented by R₁, the external fluid flow bypassing the polystyrene sulfate resin cartridge is represented by R₀ and the ratio is represented by R₁/R₀.

In any embodiment, a ratio R₁/R₀ is controlled based upon the conductivity of the dialysate or fluid.

In any embodiment, a ratio R₁/R₀ is controlled based upon an amount of urea absorbed by a sorbent cartridge.

In any embodiment, a concentration of calcium ions of a dialysate or fluid is monitored using a calcium sensing electrode, wherein a ratio R₁/R₀ is controlled based upon the concentration of calcium ions detected by the calcium sensing electrode.

In any embodiment, a ratio R₁/R₀ is controlled such that a fixed concentration of calcium ions is detected by the calcium sensing electrode.

In any embodiment, a ratio R₁/R₀ is controlled such that a concentration of calcium ions is near zero in a dialysate or fluid.

In any embodiment, at least a portion of a dialysate or fluid is conveyed through a deionization cartridge containing a mixed bed deionization resin.

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 shows a hemodialysis device having a dialysis circuit and a polystyrene sulfonate resin cartridge in a first location operating in accordance with certain embodiments.

FIG. 2 shows a hemodialysis device having a dialysis circuit, a polystyrene sulfonate resin cartridge at a first location and a deionization cartridge operating in accordance with certain embodiments.

FIG. 3 shows a hemodialysis device having a controlled compliant dialysis circuit, a polystyrene sulfonate resin cartridge at a second location and a deionization cartridge operating in accordance with certain embodiments.

FIG. 4 shows a hemodialysis device having a controlled compliant dialysis circuit, a polystyrene sulfonate resin cartridge at a third location and a deionization cartridge operating in accordance with certain embodiments.

FIG. 5 shows a hemodialysis device having a controlled compliant dialysis circuit and a polystyrene sulfonate resin cartridge at a fourth location operating in accordance with certain embodiments.

FIG. 6 shows a hemodialysis device having a controlled compliance dialysis circuit with a control reservoir, a control pump, second control reservoir and a second reservoir pump operating in accordance with certain embodiments.

FIG. 7 shows a schematic for a hematocrit detector.

FIG. 8 shows a schematic for an impedance detector.

FIG. 9 shows a schematic for a controller in communication with various system components.

### DETAILED DESCRIPTION OF THE INVENTION

A controlled compliance dialysis circuit is provided for conveying and re-circulating a dialysate between a dialyzer, where the dialysate picks up waste products or impurities such as urea, and a sorbent cartridge where waste products or impurities are removed from the dialysate to form refreshed dialysate after the addition of cation electrolytes. The dialysate flow path described herein has any one or combination of active control of fluid flow entering and exiting the flow path in a manner that allows for the accurate performance of ultrafiltration, quantization of urea removal, and the performance of convective clearance of mid-weight uremic impurities or waste products without an excessive risk for blood clotting.

### DEFINITIONS

Unless defined otherwise, all technical and scientific terms used herein generally have the same meaning as commonly understood by one of ordinary skill in the relevant art.

The articles “a” and “an” are used herein to refer to one or to more than one (i.e., to at least one) of the grammatical object of the article. By way of example, “an element” means one element or more than one element.

The term “activated carbon” refers to a porous carbon material having a surface area greater than 500 m² per gram. Activated carbon can be capable of absorbing several species including heavy metals such as lead, mercury, arsenic, cadmium, chromium and thallium among others, oxidants such as chlorine and chloramine, fluoride ions, and waste species such as phosphate and certain nitrogen-containing waste species such as creatinine and uric acid. The terms “administering,” “administer,” “delivering,” “deliver,” “introducing,”, “bolus,” and “introduce” can be used interchangeably to indicate the introduction of water or an agent into the body of a patient, including electrolytes and alkali and/or alkali earth ions, to a patient in need thereof, and can further mean the introduction of water, any agent or alkali and/or alkali earth ions to a dialysate or dialysis circuit where such water, agent or alkali and/or alkali earth ion will enter the blood of the patient by diffusion, transversal of a diffusion membrane or other means.

The term “air trap” refers to a structure for separating a gas from a mixture of a gas and a liquid. An air trap can include a hydrophobic membrane for allowing gases to pass and preventing the passage of water.

The term “anticoagulant” is a substance that prevents or delays the clotting of blood, such as heparin, fragmin, and sodium citrate.

A “biocompatible material” is a material that has the ability to interface with living biological tissues with an acceptable host response in any of specific medical systems, methods of treatment or delivery contemplated herein. The biocompatible material can consist of synthetic, natural or modified natural polymers intended to contact or interact with the biological systems during application of any of the inventions contained herein.

The term “conduit” refers to a vessel or passageway having a void volume through which a fluid can travel or move. A conduit can have a dimension parallel to the direction of travel of the fluid that is significantly longer than a dimension orthogonal to the direction of travel of the fluid.

“Chronic kidney disease” (CKD) is a condition characterized by the slow loss of kidney function over time. The most common causes of CKD are high blood pressure, diabetes, heart disease, and diseases that cause inflammation in the kidneys. Chronic kidney disease can also be caused by infections or urinary blockages. If CKD progresses, it can lead to end-stage renal disease (ESRD), where the kidneys fail to function at a sufficient level.

The terms “communicate” and “communication” include, but are not limited to, the connection of system electrical elements, either directly or remotely, for data transmission among and between said elements. The terms also include, but are not limited, to the connection of system fluid elements enabling fluid interface among and between said elements.

The term “comprising” includes, but is not limited to, whatever follows the word “comprising.” Thus, use of the term indicates that the listed elements are required or mandatory but that other elements are optional and may or may not be present.

The term “conductivity meter” or “conductivity sensor” refers to a device for measuring the electrical conductance of a solution.

The term “consisting of” includes and is limited to whatever follows the phrase “consisting of:” Thus, the phrase indicates that the limited elements are required or mandatory and that no other elements may be present.

The term “consisting essentially of” includes whatever follows the term “consisting essentially of” and additional elements, structures, acts or features that do not affect the basic operation of the apparatus, structure or method described.

The term “control pump” refers to a pump that is operable to pump fluid bi-directionally to actively control the transfer of fluid volume into or out of a compartment or circuit.

The term “control reservoir” refers to an inflexible, substantially inflexible as described herein, or flexible vessel or container accessible by the control pump that contains a variable amount of fluid.

A “control system” consists of combinations of components that act together to maintain a system to a desired set of performance specifications. The control system can use processors, memory and computer components configured to interoperate to maintain the desired performance specifications. It can also include fluid control components and solute control components as known within the art to maintain the performance specifications.

A “controller,” “control unit,” “processor,” or “microprocessor” is a device which monitors and affects the operational conditions of a given system. The operational conditions are typically referred to as output variables of the system, which can be affected by adjusting certain input variables.

The terms “controlled compliance” and “controlled compliant” describe the ability to actively control the transfer of fluid volume into or out of a compartment or circuit. In certain embodiments, the variable volume of fluid in a dialysis circuit expands and contracts via the control of one or more pumps. The volume of fluid in the system minus the attached reservoirs once the system is in operation is generally constant. The attached reservoirs allow the system to adjust the patient fluid volume by withdrawing fluid and storing the desired amount in an attached control reservoir and/or by providing rebalanced fluids to the patient and removing waste products. Alternatively, the fluid stored in a control reservoir attached to the dialysis circuit can be used for ultrafiltration (UF) and/or delivery of an infusate. The terms “controlled compliance” and “controlled compliant” are not to be confused with the term “non-compliant volume,” which simply refers to a vessel, conduit, container, pathway or cartridge that resists the introduction of a volume of fluid after air has been removed from a defined space such as a vessel, conduit, container, pathway or cartridge.

The term “convective clearance” refers to the movement of solute molecules or ions across a semi-permeable barrier due to force created by solvent molecules moving across the semi-permeable barrier.

The term “dialysate” describes a fluid into which solutes from a fluid to be dialyzed diffuse through a membrane. A dialysate typically contains electrolytes that are close in concentration to the physiological concentration of electrolytes found in blood.

“Dialysis” is a type of filtration, or a process of selective diffusion through a membrane. Dialysis removes solutes of a specific range of molecular weights via diffusion through a membrane from a fluid to be dialyzed into a dialysate. During dialysis, a fluid to be dialyzed is passed over a filter membrane, while dialysate is passed over the other side of that membrane. Dissolved solutes are transported across the filter membrane by diffusion between the fluids. The dialysate is used to remove solutes from the fluid to be dialyzed. The dialysate can also provide enrichment to the other fluid.

The term “dialysis membrane” can refer to a semi-permeable barrier selective to allow diffusion of solutes of a specific range of molecular weights through the barrier, or optionally a high permeability membrane, which is a type of semipermeable membrane that is more permeable to water than the semipermeable membrane of a conventional hemodialysis system, which has a semipermeable membrane that has a sufficiently low permeability to water such that an ultrafiltration controller is not required to prevent excessive loss of water from the patient's blood. During high permeability hemodialysis, the system removes toxins or excess fluid from the patient's blood using the principles of convection (via a high ultrafiltration rate) and/or diffusion (via a concentration gradient in dialysate). In certain non-limiting examples, the semipermeable membrane during high permeability hemodialysis has an in vitro ultrafiltration coefficient (Kuf) greater than 8 milliliters per hour per conventional millimeter of mercury, as measured with bovine or expired human blood.

The term “diluent” refers to a fluid having conductivity less than a fluid to which the diluent is added.

The term “electrolyte” refers to an alkali or alkali earth cation dissolved in an aqueous medium.

The terms “frit” and “spacer frit” refer to a material that is biocompatible and has a porosity between about 1 μm and 300 μm. The material can be one or more of biocompatible, compressible, an open cell polymer or foam or similar material.

The term “filtration” refers to a process of separating solutes from a fluid, by passing the fluid through a filter medium across which certain solutes or suspensions cannot pass. Filtration is driven by the pressure difference across the membrane.

The term “fixed volume” or “substantially inflexible volume” refers to a three-dimensional space within a vessel or container that can accommodate a maximum amount of non-compressible fluid and resists the addition of any volume of fluid above the maximum amount. The presence of a volume of fluid less than the maximum amount will fail to completely fill the vessel or container. Those skilled in the art will recognized that a fixed volume can be substantially fixed, where a minimal amount of expansion of the vessel or container can occur; however, the addition of a significant volume of fluid over a maximum will be resisted.

The term “fluid communication” refers to at least two fluids that are contained in separated compartments that are able to exchange matter, either solvent or solute molecules or ions, through a semi-permeable barrier or to allow for the movement of a fluid from one defined area to another defined area through a channel, frit, etc. separating defined areas.

“Hemofiltration” is a therapy in which blood is filtered across a semi-permeable membrane. Water and solutes are removed from the blood via pressure-driven convection across the membrane. In hemofiltration, solutes small enough to pass through the membrane in proportion to their plasma concentration are removed. The driving force is a pressure gradient rather than a concentration gradient. A positive hydrostatic pressure drives water and solutes across the filter membrane from the blood compartment to the filtrate compartment, from which it is drained. Solutes, both small and large, get dragged through the membrane at a similar rate by the flow of water that has been engineered by the hydrostatic pressure. Hence, convection overcomes the reduced removal rate of larger solutes (due to their slow speed of diffusion) seen in hemodialysis. The rate of solute removal is proportional to the amount of fluid removed from the blood circuit, which can be adjusted to meet the needs of a clinical situation. In general, the removal of large amounts of plasma water from the patient requires volume substitution. Substitution fluid, typically a buffered solution close to the plasma water composition a patient needs, can be administered pre or post filter (pre-dilution mode, post-dilution mode).

“Hemodialysis” is a technique where blood and a “cleansing fluid” called dialysate are exposed to each other separated by a semi-permeable membrane. Solutes within the permeability range of the membrane pass while diffusing along existing concentration gradients. The dialysate employed during hemodialysis has soluble ions such as sodium, calcium and potassium ions and is not pure water. The sieving properties of the membrane exclude all solutes above a certain threshold from crossing the membrane. One common sieving property is “albumin sieving.” In most situations, it is not desirable to remove Albumin during renal replacement therapy, as lower blood serum Albumin is associated with increased mortality rates.

The term “albumin sieving coefficient” can be used to describe the amount of albumin that will cross the membrane.

The term “hematocrit” refers to the fraction of blood volume occupied by erythrocytes.

“Hemodiafiltration” is a therapy that combines hemofiltration and hemodialysis.

The term “impedance meter” refers to a device for measuring the opposition of an object or structure to an alternating current.

The term “infusate container” refers to a vessel, which can be inflexible or flexible, for holding a solution of one or more salts for the adjustment of the composition of a dialysate.

The term “infusate solution” refers to a solution of one or more salts for the adjustment of the composition of a dialysate, such as salts of calcium, magnesium and potassium.

The term “impurity species” refers to a molecular or ionic species that originates from tap water, a sorbent cartridge or a source other than a patient's or subject's blood including chlorine, fluoride ions, and aluminum-containing species.

The term “nitrogenous waste” refers to any non-polymeric nitrogen-containing organic compound originating from the blood of a patient. Nitrogenous waste includes urea and creatinine.

The term “waste species” or “waste products” refer to any molecular or ionic species originating from the patient or subject, including metabolic wastes, molecular or ionic species including nitrogen or sulfur atoms, mid-weight uremic wastes and nitrogenous waste. Waste species are kept within a specific homeostasis range by individuals with a healthy renal system. For example, nitrogen-containing waste products are generally at a level less than 30 mg/dL in the blood for individuals with a healthy renal system and inorganic phosphate can be generally in a range between 2.5-4.5 mg/dL but not necessarily limited to this range. The level of waste products in the blood is elevated for individuals with impaired kidney function.

The term “non-compliant volume” refers to a vessel, conduit, container, pathway or cartridge that resists the introduction of a volume of fluid after air has been removed from a defined space such as a vessel, conduit, container, pathway or cartridge.

The term “oximeter” refers to a device for measuring the amount of oxygen carried by a volume of blood.

The term “predominately” or “predominately containing” in relation to the structure of a polystyrene sulfonate resin refers to a polystyrene sulfonate resin wherein the most prevalent individual repeat unit by mole fraction forming the polystyrene sulfonate resin originates from a polystyrene sulfonate monomer, wherein the polystyrene sulfonate repeat unit does not need to form the majority of the polystyrene sulfonate resin by mole fraction. For example, a polystyrene sulfonate resin predominately containing a polystyrene sulfonate monomer can contain 40% polystyrene sulfonate repeat units, 20% repeat units of a second type, 20% repeat units of a third type and 20% repeat units of a fourth type.

The term “luer connector” or “luer adapter” refers to adapters or connector conforming with International Standards Organization (ISO) standards 594.

The term “conical” refers to a shape for a cartridge or container that has a “tapered shape” as defined herein, wherein the diameter for a cross-section taken perpendicular to an axis of the cartridge or container, where the diameter of the cross-section changes for translation along the axis from a smaller diameter to a larger diameter or from a larger diameter to a smaller diameter.

The term “cylindrical” refers to a shape for a cartridge or container that has a substantially uniform diameter for a cross-section taken perpendicular to an axis of the cartridge or container.

The term “memory” refers to a device for recording digital information that can be accessed by a microprocessor, such as RAM, Dynamic RAM, microprocessor cache, FLASH memory, or memory card.

The term “mid-weight uremic waste” refers to substances that can pass through a dialysis membrane and that have a molecular weight less than about 66000 g/mol and greater than about 1000 g/mol.

The term “moving fluid bi-directionally” refers to the ability to move a fluid across a barrier, such as a semi-permeable membrane, in either direction through the thickness of the barrier.

“Osmolarity” is defined as the number of osmoles of a solute per liter of solution. Thus, a “hyperosmolar solution” represents a solution with an increase in osmolarity compared to physiologic solutions. Certain compounds, such as mannitol, may have an effect on the osmotic properties of a solution as described herein.

A “patient” or “subject” is a member of any animal species, preferably a mammalian species, optionally a human. The subject can be an apparently healthy individual, an individual suffering from a disease, or an individual being treated for a disease.

The terms “pathway” and “conveyance pathway” refer to the route through which a fluid, such as dialysate or blood, travels.

The term “peristaltic pump” refers to a pump that operates by compression of a flexible conduit or tube through which the fluid to be pumped passes.

The terms “portable system” or “wearable system” refers to a system in whole or in part having a mass and dimension to allow for transport by a single individual by carrying the system or wearing the system on the individual's body. The terms are to be interpreted broadly without any limitation as to size, weight, length of time carried, comfort, ease of use, and specific use by any person whether man, woman or child. The term is to be used in a general sense wherein one of ordinary skill will understand that portability as contemplated by the invention encompasses a wide range of weights, geometries, configurations and size.

The terms “pressure differential” and “pressure drop” refer to the difference in pressure measurements of a fluid between two points of measurement.

The term “pressure meter” refers to a device for measuring the pressure of a gas or liquid in a vessel or container.

The terms “processor,” “computer processor,” and “microprocessor” as used herein are broad terms and are to be given their ordinary and customary meaning to a person of ordinary skill in the art. The terms refer without limitation to a computer system, state machine, processor, or the like designed to perform arithmetic or logic operations using logic circuitry that responds to and processes the basic instructions that drive a computer. In some embodiments, the terms can include ROM (“read-only memory”) and/or RAM (“random-access memory”) associated therewith.

The term “programmable” as used herein refers to a device using computer hardware architecture with a stored program and being capable of carrying out a set of commands, automatically that can be changed or replaced.

The term “pulsatile pump” refers to a pump that mimics the action of a mammalian heart where the pumped fluid undergoes periodic variation in velocity.

The term “pump” refers to a device that causes the movement of fluids or gases by the application of suction or pressure.

The term “quick connector” refers to any structure for making an attachment that is operable by an individual using their hands or fingers without the assistance of additional tools. The quick connector can have a valve that shuts off flow when the connector is disconnected.

The term “relative blood volume monitor” refers to any device measuring the concentration of any solute or solid material in the blood. Non-limiting examples of relative blood volume monitors include devices for measuring the concentration of oxyhemoglobin, deoxyhemoglobin, hematocrit or red blood cell count, osmolarity or total protein concentration of the blood.

The term “relative blood volume hydration status” refers to the relative change in the level of any target solute or solid material in the blood over a period of time. Non-limiting examples of target solute or solid materials include oxyhemoglobin, deoxyhemoglobin, hematocrit or red blood cell count, osmolarity or total protein concentration of the blood. Relative blood volume hydration status can be monitored by observation of a change in a signal responsive to the level of any target solute or solid material in the blood without a requirement that the absolute concentration of the target solute or solid material be determined.

The term “spent dialysate” refers to a dialysate that has been contacted with blood through a dialysis membrane and contains one or more impurity or waste species or waste substance, such as urea.

The term “sorbent cartridge” refers to a cartridge containing one or more sorbent materials for removing specific solutes from solution, such as urea.

The terms “treating” and “treatment” refer to the management and care of a patient having a pathology or condition by administration of one or more therapy contemplated by the present invention. Treating also includes administering one or more methods of the present invention or using any of the systems, devices or compositions of the present invention in the treatment of a patient. As used herein, “treatment” or “therapy” refers to both therapeutic treatment and prophylactic or preventative measures. “Treating” or “treatment” does not require complete alleviation of signs or symptoms, does not require a cure, and includes protocols having only a marginal or incomplete effect on a patient.

The term “ultrafiltration” refers to subjecting a fluid to filtration, where the filtered material is very small; typically, the fluid comprises colloidal, dissolved solutes or very fine solid materials, and the filter is a microporous, nanoporous, or semi-permeable medium. A typical medium is a membrane. During ultrafiltration, a “filtrate” or “ultrafiltrate” that passes through the filter medium is separated from a feed fluid. In general, when transport across a membrane is predominantly diffusive as a result of a concentration driving force, the process is described herein as dialysis. When transport is primarily convective as a result of bulk flow across the membrane induced by a pressure driving force, the process is ultrafiltration or hemofiltration depending on the need for substitution solution as the membrane passes small solutes but rejects macromolecules. The term “ultrafiltration” can also refer to the fluid removal from blood during a dialysis or a hemofiltration process. That is, ultrafiltration refers to the process of passing fluid through a selective membrane, such as a dialysis or hemofiltration membrane, in either a dialysis, hemodiafiltration or filtration process.

The term “void volume” refers to a specific volume that can be occupied by a fluid in a defined space such as a dialysis circuit of the invention including all components contained therein.

“Diffusive permeability” is a property of a membrane describing permeation by diffusion. Diffusion is the process of solutes moving from an area of higher concentration to an area of lower concentration

The term “porosity,” as used herein describes the fraction of open pore volume of a membrane.

The term “shunt,” as used herein describes a passage between channels, such as blood vessels, where the shunt diverts or permits flow from one pathway or region to another.

The term “plumbing,” as used herein generally describes any system of valves, conduits, channels, and lines for supplying any of the fluids used in the invention.

The term “extracorporeal,” as used herein means situated or occurring outside the body.

The term “effluent dialysate,” as used herein describes the discharge or outflow after the dialysate has been used for dialysis.

The term “metabolic waste species,” as used herein describes organic and inorganic components generated by a patient. They can be metabolic products such as urea, uric acid, creatinine, chlorides, inorganic sulfates and phosphate, or excess electrolytes such as sodium, potassium, etc. It will be understood that the specific “metabolic waste species” can vary between individuals depending on diet and environmental factors. Hence, the term is intended to encompass any waste component that is normally removed by a kidney or by dialysis without restriction on the specific type of waste substance.

The term “metabolic waste species,” as used herein describes organic and inorganic components generated by a patient. They can be metabolic products such as urea, uric acid, creatinine, chlorides, inorganic sulfates and phosphate, or excess electrolytes such as sodium, potassium, etc. It will be understood that the specific “metabolic waste species” can vary between individuals depending on diet and environmental factors. Hence, the term is intended to encompass any waste component that is normally removed by a kidney or by dialysis without restriction on the specific type of waste substance.

The term “working dialysate solution” refers to a dialysate solution that is undergoing active circulation or movement through a system including conduits, pathways, dialyzers and cartridges.

### Sorbents for Dialysis Regeneration

Dialysate eluted from a dialyzer employed during hemodialysis therapy contains impurities and waste products, such as urea, that entered the dialysate from the blood or from other external sources such as a water source for dialysate generation or leaching from the sorbent cartridge itself. The sorbent cartridge can remove these impurities and waste products prior to reuse of the refreshed dialysate. Sorbent materials that can perform removal of impurities and waste products and regenerate the dialysate for use in the controlled compliance dialysis circuit are known. Examples of useful sorbent materials include the REDY sorbent system. The sorbent cartridge typically contains four different kinds of materials as follows: 1) a urease-containing material, where urease is an enzyme that catalyzes the conversion of urea to ammonia (ammonium ions) and carbon dioxide; 2) a zirconium phosphate (ZrP) material that has the capacity to act as a cation exchanger by absorbing a large quantity of ammonium ions in exchange for sodium and hydrogen ions, where the ZrP material also exchanges Mg²⁺, Ca²⁺ and K⁺ ions for sodium and hydrogen ions; 3) a zirconium oxide material (ZrO), which acts as an anion exchanger by exchanging phosphate for acetate and bicarbonate; and 4) an activated carbon material that has a surface area for adsorption of a wide range of impurities and waste products including metal ions and uremic toxins, such as uric acid, creatinine, and β2-microglobin. Examples of useful sorbent materials include the REDY sorbent system and U.S. Pat. Nos. 3,669,880; 3,989,622; 4,581,141; 4,460,555; 4,650,587; 3,850,835; 6,627,164; 6,818,196; and 7,566,432 and U.S. Patent Publications 2010/007838; 2010/0084330; and 2010/0078381, which are incorporated herein by reference. In certain embodiments, the urease-containing material, the zirconium phosphate material, the zirconium oxide material, and the activated carbon material are arranged into discrete layers within the sorbent cartridge. The various sorbent materials can be provided in separate housings or as discrete layers within such housings in certain embodiments. In certain other embodiments, the urease-containing material and the zirconium phosphate material are intermixed in the same discrete layer within the sorbent cartridge. The urease-containing material can be immobilized or covalently linked to a substrate material. The substrate material is not particularly limited, where suitable substrate materials include organic polymers, carbohydrate-based polymers, polyamides, polyesters, inorganic polymeric materials, chitosal and silica gel. The inclusion of the urease-containing material and the zirconium phosphate material in the same discrete layer can improve workability of the sorbent materials to prevent clogging of the sorbent cartridge or improve absorption of ammonium ions by the zirconium phosphate material. Further, zirconium phosphate materials can be replaced with magnesium phosphate materials as shown in U.S. Pat. Nos. 4,460,555 and U.S. Pat. No. 2,650,587, which are incorporated herein by reference.

Disclosed herein are systems, materials and methods for removing sodium, phosphates, sulfate and carbonate ions from the dialysate in a manner that does not exchange an ionic species removed from the dialysate with a replacement species. Specifically, systems, materials and methods are disclosed for the removal of phosphates, sulfate and carbonate ions by precipitation in lieu of ion exchange. Systems, materials and methods are further provided that remove sodium ions from the dialysate in response to the conductivity of the dialysate.

### Exemplary Dialysis Flow Path

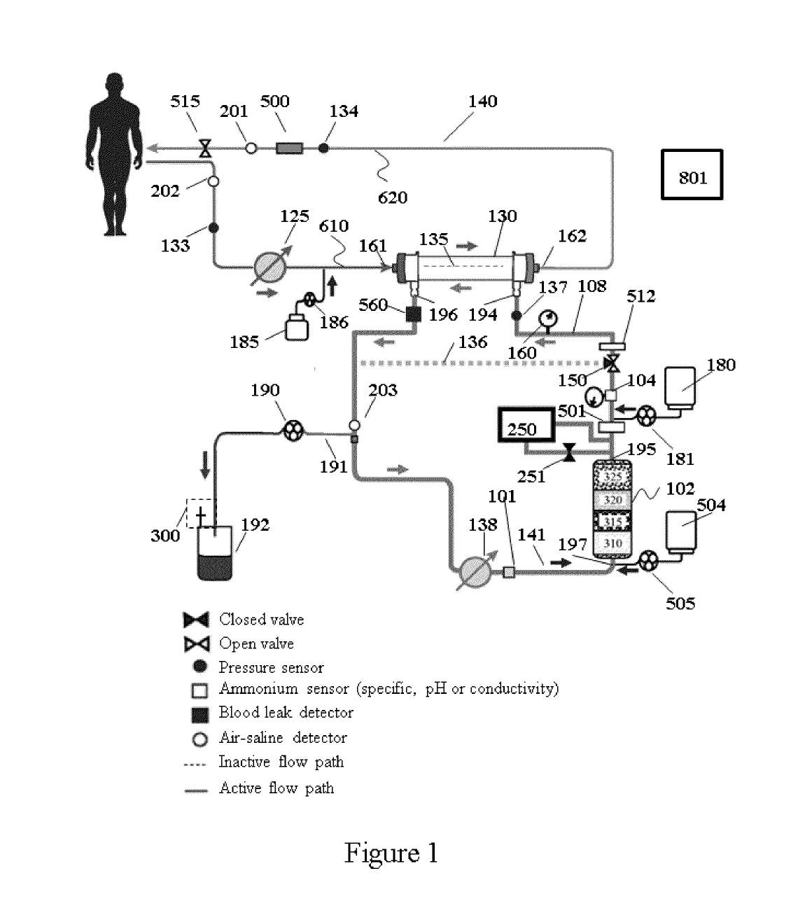

FIG. 1 shows a system and flow path for circulating blood and a dialysate through a dialyzer 130. A shunt, such as a needle or catheter, is connected to a patient's vasculature to draw blood and circulate the patient's blood through an extracorporeal circuit 140. Access to the shunt can be controlled by a valve 515. The portion of the extracorporeal circuit 140 that contains drawn blood from the patient can be referred to as the arterial line 610, which by convention is understood to mean a line for transporting blood from the patient regardless of whether blood is drawn from an artery or vein of the patient, and the portion that returns blood to the patient can be referred to as the venous line 620. In certain embodiments, the arterial line 610 and the venous line 620 connect with one or more veins of the patient. Locomotive power for moving the blood through the extracorporeal circuit 140 is provided by a blood pump 125, which is typically located along the arterial 610 line. Blood is typically conveyed through the extracorporeal circuit 140 at a rate of 50 to 600 mL/min and can be adjusted by a controller to any required rate suitable for a procedure performed by the invention. Blood pump 125 can be a peristaltic pump, although those skilled in the art will readily understand that other types of pumps can be used, including diaphragm pumps, centrifugal pumps, and shuttle pumps. In certain embodiments, the blood pump 125 conveys blood through the dialyzer 130 where the blood is contacted with a blood side of a high permeability dialysis membrane 135. Blood enters the dialyzer 130 through a blood inlet 161 and exits through a blood outlet 162. The pressure of the blood prior to the dialyzer 130 is measured by a pressure meter 133 and post dialyzer 130 by a pressure meter 134. An air trap 500 is placed along the extracorporeal circuit 140 to prevent the introduction of air into the circulatory system of the patient. The air trap 500 is not limited to a particular design. Typical air traps employ a hydrophobic membrane that allows air to be separated from an air-liquid mixture by allowing air to pass through the membrane and retaining water-based fluids. Alternatively the air trap may be run full and the pressure sensor uses a flexible impermeable membrane to transmit pressure pulses to the pressure transducer so there is no direct air blood interface. Air-fluid detectors 201 and 202 are present to confirm that air is not present in the extracorporeal circuit 140. Air-fluid detectors 201 and 202 can be ultrasonic sensors that can detect a change in solution density or scattering due the presence of air or air bubbles.

During the course of conveyance of blood along the extracorporeal circuit 140, heparin or other anticoagulant is added to the blood to prevent clotting of blood within the dialyzer 130 or blood conveyance pathway/extracorporeal circuit 140. Heparin or another anticoagulant is added from an anticoagulant container 185 at a metered rate using an anticoagulant pump 186. The anticoagulant pump 186 can be any pump capable of accurately metering heparin. Alternatively, a surface of the extracorporeal circuit 140 can be covalently bound to heparin or a like anticoagulant.

Dialysate within the system is conveyed through one of a first dialysate pathway 108 in the dialysis circuit 141, which carries dialysate to the dialyzer 130, or a second bypass pathway 136 shown in a dashed line, which serves to bypass the dialyzer 130. The first and second pathways 108 and 136 have one or more conduits for conveying the dialysate. Access to the second bypass pathway 136 is controlled by valve 150. It is understood by one skilled in the art that three-way valve 150 can be replaced with a two-way valve with the same result to control the flow through the dialyzer 130 or bypass pathway 136. The first dialysate pathway 108, the second bypass pathway 136, and residual volume in the dialyzer 130 including conduits for conveying the dialysate together form a dialysis circuit 141 that houses the circulating volume of the dialysate present in the system.

Dialysate that is conveyed through the dialyzer 130 on the dialysate side of the dialysis membrane 135 picks up waste products from the blood, including urea, by diffusion, hemofiltration or hemodiafiltration. Dialysate enters the dialyzer at a dialysate inlet end 194 and exits at an outlet end 196 The dialysate exiting the dialyzer 130 passes through a blood leak detector 560 that can determine the presence of blood in the dialysate indicating a breach in the dialysis membrane 135. Flow of dialysate from the dialyzer 130 can be stopped or controlled through the operation of valve 158. Valve 158 can also be operated to prevent the backup of dialysate into the dialyzer 130. The dialysate is conveyed through a sorbent cartridge 102 to remove waste products before being re-conveyed through the dialyzer 130. The dialysate enters the sorbent cartridge 102 at a dialysate inlet end 197 and exits at an outlet end 195. An air trap 501 can be positioned before or after outlet end 195 to remove gasses introduced into the dialysate by the sorbent cartridge 102. The volume of actively circulating dialysate is determined by the total void volume of the conduits and the sorbent cartridge 102 forming the dialysis circuit 141. The void volumes of the conduits and of the sorbent cartridge 102 forming the dialysis circuit 141 have a substantially inflexible volume.

In certain embodiments, the dialysis circuit 141 has a void volume from about 0.15 to about 0.5 L or from about 0.15 to about 1 L. In other embodiments, the dialysis circuit 141 has a void volume from about 0.2 to about 0.4 L or from 0.2 to about 0.35 L. Other volumes can be envisioned by those of ordinary skill in the art depending on parameters such as patient weight, size, and health condition. The system can be designed to be a portable system, a desktop system or a large system suitable for heavy use in a clinical setting. Hence, both large volumes greater than 0.5 to about 5 L, and micro-volumes from as small as 0.1 to about 0.5 L such as 0.1 to 0.2, 0.1 to 0.3, 0.1 to 0.4, 0.2 to 0.3, 0.3 to 0.4, 0.3 to 0.5 or 0.6 to 1 L are contemplated by the invention.

The flow path for the dialysis circuit 141 defines a controlled compliance dialysis circuit. In the controlled compliance dialysis circuit disclosed herein, passive movement of fluid across the dialysis membrane due to operational pressure change is eliminated. The invention provides for the ability to accurately control net patient fluid status, and/or diffusion combined with increased clearance via convection, and/or active provisioning of extra fluid to a patient. The invention can actively provide fluid to the patient when the patient becomes hypotensive or hypovolemic, and replace a blood circuit with a physiological solution when a patient is taken off a system. The invention can also provide for adjusting convective clearance. Any combination of the above mentioned features is contemplated by the invention. The system allows for the return of blood from the system back to the patient without necessarily providing for additional fluids. The system can optionally account for an infusate volume, provide additional convective clearance, and/or provide control of the entire process. In contrast, non-expandable volume systems do not allow for ultrafiltration (UF), the ability to give fluid to the patient and convective clearance. Instead of a fixed or non-compliant volume used in known systems, the present invention can intentionally change a volume to push fluid to or from the patient where the system controls volume and compliance (both of which are dynamic and changing) to achieve the desired goals of a therapy. The controlled compliance dialysis circuit also simplifies the entire system. Specifically, scales or gravimetric methods are not required to balance fluid removal with fluid replacement.

The controlled compliance dialysis circuit has two points where fluid can enter the dialysate flow path: 1) infusate pumps and 2) a control pump that controls the movement of fluid across the dialysis membrane. The controlled compliance dialysis circuit operates by employing two principal components: 1) an extracorporeal circuit that is attached to the vasculature and the circulation of a patient, and 2) a dialysis circuit having a limited void volume for the circulation of a dialysate. The extracorporeal circuit is an extension of the patient's circulatory system external to the patient's body. Any fluid added to the dialysis circuit 141 can enter the patient's body; likewise, any fluid drawn out of the extracorporeal circuit 140 can originate from the patient's body. Due to the connection between the extracorporeal circuit 140 and the vascular system, there is freedom of movement for fluid to flow into and out of the extracorporeal circuit due to the relatively large volume of the patient's body to accommodate an influx of fluid or to serve as a reservoir for fluid. As will be described in greater detail below, a control pump 190 is employed to actively control fluid movement between the extracorporeal circuit 140 and the dialysis circuit 141. This capability is used to enhance the convective clearance of the system while controlling the net fluid removed from the patient.

The total void volume of the conduits having a substantially inflexible volume prevents the passive inflow and outflow of fluid volume due to pressure changes that can occur over the course of treatment. This is beneficial because not all pressure changes during treatment are under precise control by a user or operator. A controlled compliance dialysis circuit is achieved by actively controlling the inflow (influx) and outflow (efflux) of fluid to and from the dialysis circuit 141 and the extracorporeal circuit 140. In this manner, the volume of fluid crossing the dialysate membrane 135 is under direct control and can be accurately determined. In certain embodiments, the dialysis circuit 141 has a void volume from about 0.15 to about 0.5 L or from 0.15 to about 1 L. In other embodiments, the dialysis circuit 141 has a void volume from about 0.2 to about 0.4 L or from 0.2 to about 0.35 L. Other volumes can be envisioned by those of ordinary skill in the art depending on parameters such as patient weight, size, and health condition. The system can be designed to be a portable system, a desktop system or a large system suitable for heavy use in a clinical setting. Hence, both large volumes greater than 0.5 to about 5 L, and micro-volumes from as small as 0.1 to about 0.2 L are contemplated by the invention.

The controlled compliance dialysis circuit can be accurately controlled to precisely remove or add fluid to the dialysis circuit 141. Due to the fixed void volume of the conduits, the sorbent cartridge 102 and other components of the dialysis circuit 141, the net movement of fluid over any time interval across the dialysate membrane 135 can be accurately controlled by creating a means to accurately introduce or remove fluid from the patient.

The controlled compliance dialysis circuit can also control the movement of fluid across the dialysis membrane 135 without affecting the flow rate of dialysate entering the dialyzer 130. Systems that rely solely on the internal pressure of the dialysis circuit to perform ultrafiltration have the disadvantage that the return of dialysate to the dialyzer is reduced by the amount of fluid being removed by ultrafiltration. The rate at which ultrafiltered fluid leaves the dialysis circuit necessarily lowers the return flow of dialysate to the dialyzer. In contrast, the present invention contemplates a separate control pump 190 and dialysate pump 138 that allow for the rate of fluid return to the dialyzer to remain constant and/or not affected by the removal of fluid volume from the dialysis circuit.

As shown in FIG. 1, the dialysate is moved along the dialysis circuit 141 by a dialysate pump 138. When the control pump 190 is not operating, fluid along the length of the dialysis circuit 141 flows at a rate determined by the dialysate pump 138. When the control pump 190 is operating, fluid exiting the dialyzer 130 and traveling toward the conduit 191 is flowing at a rate that is the combination of the rates of the control pump 190 and the dialysate pump 138. However, the fluid traveling from the entry point of conduit 191 into the dialysis circuit 140 to the dialyzer 130 is traveling at the rate of the dialysate pump 138. The rate of fluid traveling to the dialyzer 130 is not affected by the operation of the control pump 190. The dialysate pump can be operated at a rate from about 10 to about 400 mL/min, the specific rate being dependant on the rate of the blood pump 125 at the desired contact time with the dialysis membrane 135 to achieve diffusion of impurities from blood to the dialysate. The rate of the dialysate pump 138 and the blood pump 125 can be controlled by a controller 801.

Refreshed dialysate exiting an outlet end of the sorbent cartridge 102 can be monitored by a conductivity meter 104 and/or a conductivity meter 160. The design of any conductivity meter employed in embodiments described herein is not particularly limited; however, a typical conductivity meter has two electrodes where a current between the two electrodes is monitored. The presence of sodium ions in the dialysate is the major contributor to the conductivity measured by conductivity meter 104. Conductivity is continually monitored and reported to the controller to assess the quality and safety of the dialysate. When the conductivity of the dialysate falls within a predetermined range, the dialysate is directed by valve 150 to a dialysate inlet end 194 of the dialyzer 130; the valve 150 is located between an outlet end 195 of the sorbent cartridge 102 and the dialysate inlet end 194 of the dialyzer 130. In certain embodiments, the valve 150 is a three-way valve. Optionally, the dialysate can be filtered through a microbial filter 512. The pressure of the dialysate entering the dialysate inlet end of the dialyzer 130 is measured by a pressure meter 137. In certain embodiments, the predetermined range for the conductivity of the dialysate is from about 12.6 to about 15.4 mS/cm.

When the conductivity measured by meter 104 is outside of the predetermined range, the valve 150 directs the dialysate to be conveyed through the second dialysis flow path 136 shown as a dashed line Further, valve 158 can be closed to prevent the dialysate from backing up into the dialyzer 130. The dialysate can be circulated through the sorbent cartridge 102 while bypassing the dialyzer 130 and preventing contact with the patient's blood when required. Since the dialysis circuit 141 is isolated from the extracorporeal circuit 140 when valve 158 is closed, the control pump 190 is not operated when valve 158 is in a closed position during normal operation. When the system is being primed, control pump 190 can operate to vent air from the dialysis circuit 141, as described below.

Due to the substantially inflexible volume of the conduits and the sorbent cartridge 102, bulk fluid or water is prevented from moving from across the membrane 135 from the extracorporeal circuit 140 of the dialyzer 130 to the dialysis circuit 141 of the dialyzer 130. Specifically, due to the inflexible void volume of the dialysis circuit 141, water cannot passively move from the extracorporeal side to the dialysate side through the dialysis membrane. In the event of factors that tend to increase pressure on the extracorporeal side of the dialysis membrane, such as increased blood flow rate or blood viscosity, pressure across the membrane will automatically be equalized due to the limited volume of the dialysis circuit 141 and the non-compressible nature of the dialysate. In the event of factors that tend to increase pressure on the dialysate side of the dialysis membrane 135, such as increased dialysis flow rate, net movement of water from the dialysis circuit 141 to the extracorporeal circuit 140 is prevented by a vacuum that would form in the dialysis circuit 141 in the event of such a movement. Since the dialyzer is a high-flux type there is some fluid flux back and forth across the dialyzer membrane due to the pressure differential on the blood and dialysate sides of the membrane. This is a localized phenomenon due to the low pressure required to move solution across the membrane and is called backfiltration, however results in no net fluid gain or loss by the patient.

Using the controlled compliance dialysis circuit described herein, net movement of water across the dialysis membrane occurs under active control rather than passively due to pressure differences that develop across the dialysis membrane due to normal operations. A control pump 190 is present and accesses the controlled compliance dialysis circuit 141 through a conduit 191. In certain embodiments, the conduit 191 joins with the controlled compliance dialysis circuit 141 at a point downstream from the dialyzer 130. The control pump 190 can be operated in an influx direction that moves fluid from a control reservoir 192 to the controlled compliance dialysis circuit 141 or in an efflux direction that moves fluid from the controlled compliance dialysis circuit 141 into the control reservoir 192. Due to the fixed volume of the dialysis circuit 141, volume added to the controlled compliance dialysis circuit when the control pump 190 operates in the influx direction causes net movement of fluid from the dialysate side of the dialysis membrane 135 to the extracorporeal side of the dialysis membrane 135. When the compliance control pump 190 is operated in the efflux direction, fluid is drawn from the extracorporeal side of the dialysis membrane into the controlled compliance dialysis circuit. In certain embodiments, the control pump 190 can be operated at a rate from 0 to about 200 mL/min in either direction. In certain other embodiments, the control pump 190 can be operated at a rate from 0 to about 100 mL/min or 0 to 50 mL/min in either direction. In certain other embodiments, the control pump 190 can be operated at a rate from 0 to about 100 mL/min or 0 to 50 mL/min in either direction. Any range from about 0 to about 200 mL/min is contemplated by the invention such as about 15 to about 185 mL/min, about 25 to about 175 mL/min, about 5 to about 75 mL/min, about 61 to about 183 mL/min, about 156 to about 193 mL/min, about 32 to about 63 mL/min, about 145 to about 199 mL/min, about 16 to about 93 mL/min or, about 29 to about 124 mL/min.

In embodiments where the control pump 190 is operated in the influx direction, the dialysate pump 138 operates at a rate higher than the control pump 190 to prevent flow of the used dialysate back into the dialyzer 130. The dialysate pump 138 functions to convey the dialysate from the point where line 191 joins the dialysis circuit 141 to the sorbent cartridge 102. A rate of the dialysate pump 138 operating faster than the control pump 191 in the influx direction ensures that the contents of the control reservoir 192 are conveyed to the sorbent cartridge 102 and do not reach the dialyzer 130 without first passing through the sorbent cartridge 102. In certain embodiments, the dialysate pump 138 operates at a rate that is about 100 mL/min greater and at rates greater than the rate of the control pump 190, when the control pump 190 is operating in the influx direction. For example, if the rate of the control pump 190 is 10 mL/min, the dialysate pump 138 can operate at rates greater than about 110 mL/min such as 130 mL/min, 175 mL/min, 210 mL/min, 510 mL/min, 760 mL/min, 1 L/min, 1.6 L/min. If the rate of the control pump 190 is 25 mL/min, the dialysate pump 138 can operate at rates greater than about 125 mL/min such as 130 mL/min, 175 mL/min, 210 mL/min, 510 mL/min, 760 mL/min, 1 L/min, 1.6 L/min. In one embodiment, the dialysate pump 138 operates at a rate that is about 20 mL/min greater and at rates greater than the rate of the control pump 190 or higher, when the control pump 190 is operating in the influx direction. In other embodiments, the dialysate pump 138 operates at a rate that is about twice the rate and at rates greater than that of the control pump 190, when the control pump 190 is operating in the influx direction. In certain embodiments, the dialysate pump 138 operates at a rate that is about 5% higher and at rates higher than the rate of the control pump 190, when the control pump 190 is operating in the influx direction. For example, the dialysate pump 138 can operate at 6%, 7%, 8%, 10%, 15%, 45%, 63%, 75%, 100%, 200%, 500%, 2000%, or any higher percentage than the rate of the control pump 190.

The control reservoir 192 is not limited to any particular structure. In certain embodiments, the control reservoir 192 can be made from a flexible or collapsible material that expands depending on the volume held. In certain embodiments, the control reservoir 192 can be substantially inflexible. The control reservoir 192 can include a hydrophobic 0.2 micron (μm) sterile, non-pyrogenic, and non-toxic air filter 300 to prevent the entry of bacteria or endotoxin into the control reservoir 192 and dialysis circuit 141. Further, air filter 300 can also release air pressure present in the control reservoir 192. The material of air filter 300 may be Millipore Dualex™ filter or an equivalent. In certain embodiments, the control reservoir 192 can have a valve that allows the patient to empty the volume of the control reservoir 192 without interrupting treatment.

Several sensors and monitors can be employed to determine the state of the dialysis system, as shown in FIG. 1. Blood leaks across the dialysis membrane 135 can be detected by a blood leak detector 560. The blood leak detector 560 can be an optical detector having a light source and photo detector allowing for the observation of a red color in the dialysate. The presence of air or fluid in the extracorporeal 140 and dialysis circuits 141 can be determined by air-fluid detectors 201, 202, and 203 which can be ultrasonic sensors that can detect a change in solution density or scattering due the presence of air or air bubbles. Conductivity meters 101, 104 and 160 can be present to monitor the composition of the dialysate within the dialysis circuit. Pressure meters 133, 134 and 137 can be present to determine and unsafe operating pressure and/or fluid leak from the system. The pressure at pressure meter 133 gives an indication of the adequacy of the blood flow into the circuit, increased vacuum is an indication of a less adequate access flow. The pressure indication at pressure meter 134 indicates obstructions in the venous bloodline. The pressure meter can be a transducer device that operates through capacitive or piezoelectric principals to convert the amount of force applied to a surface to an electronic signal.

The dialysis systems described herein, including the system described in FIG. 1, can have a use for various novel methods of kidney replacement therapy. The dialysis systems have an extracorporeal circuit 140 having a first end that draws blood from the patient and a second end that returns blood to the patient, and a dialysis circuit 141. The extracorporeal circuit 140 and the dialysis circuit 141 are in fluid communication through a dialysis membrane 135 housed in a dialyzer 130. The systems have a configuration to perform novel methods of kidney replacement therapy as described herein.

### Polystyrene Sulfonate Resin

As described above, the use of sorbent materials that act through ion exchange create changes in the composition of the dialysate that require constant or periodic adjustments to maintain the dialysate in a useable state. Specifically, in the processes of removing urea and phosphate from the dialysate by ion exchange mechanisms, additional species such as sodium, carbonate and acetate are added to the dialysate that in turn have to be removed from the dialysate in order to maintain the composition of the dialysate.

Over the course of treatment, ammonium ions generated from urea are absorbed by the sorbent cartridge 102 and sodium ions and hydrogen ions are simultaneously released. In order to address the increase in conductivity of the dialysate created by the addition of sodium ions by ion exchange, traditional systems actively add a low-conductivity diluent to the dialysate to dilute sodium ion concentration. However, such methods involve adding additional components to the system that increase the weight of the system, including a significant weight of diluent, and thereby decrease the wearability and portability of the hemodialysis system.

In any embodiment of the present invention, the sodium chloride concentration is controlled through the use of a polystyrene sulfonate resin contained in a polystyrene sulfonate resin cartridge that operates to remove sodium ions in exchange for calcium ions. The polystyrene sulfonate resin cartridge contains the polystyrene sulfonate resin. Within the polystyrene sulfonate resin cartridge, the released calcium ions react with carbonate ions, sulfate ions and/or phosphates present in the dialysate to form a solid precipitate that remains within the cartridge. As such, the polystyrene sulfonate resin cartridge can be used to remove several species from the dialysate without adding any undesirable species to the dialysate. Specifically, the polystyrene sulfonate resin cartridge can remove sodium ions, phosphates, carbonate ions and sulfate ions from the dialysate without adding any additional species to the dialysate unlike traditional ion exchange materials. While most of the exchanged calcium ions will form a solid precipitate, calcium ions that escape the polystyrene sulfonate resin cartridge can alleviate the need to infuse calcium ions into the dialysate.

In any embodiment of the present invention, the polystyrene sulfonate resin cartridge can reduce or replace the need for an expensive zirconium oxide material within the sorbent cartridge 102. The zirconium oxide material functions to absorb phosphates, which is a function that is also performed by the polystyrene sulfonate resin cartridge. The polystyrene sulfonate resin is capable of undergoing the reaction in Equation (1):

(RX⁻)₂Ca²⁺+Na⁺(aq)→2RX⁻Na⁺+Ca²⁺(aq) (1)

As shown in Equation (1), RX⁻ represents a moiety that releases a calcium ion in exchange for two sodium ions, where X is any appropriate functional group for coordinating calcium ions and R is an organic group. The stoichiometry of Equation (1) assumes that the moiety RX⁻ has a single negative charge; those skilled in the art will understand that the stoichiometry of Equation (1) is modified if the moiety has a multiple valence. The released calcium ions are capable of precipitating carbonate ions, sulfates and phosphates, as shown in Equations (2) through (4), respectively, where x is an integer from 0 to 2:

Ca²⁺(aq)+CO₃²⁻(aq)→CaCO₃(s) (2).

Ca²⁺(aq)+SO₄²⁻(aq)→CaSO₄(s) (3).

Ca²⁺(aq)+HₓPO₄^{x-3}(aq)→Ca₃(PO₄)₂(s)+xH⁺ (4),

In certain embodiments, the polystyrene sulfonate resin predominately contains the structure of Formula (I), which is a resin based upon polystyrene sulfonate (sulfonated polystyrene). The variable “n” in Formula (I) in not particularly limited; however, in certain embodiments “n” is from about 5 to about 200.

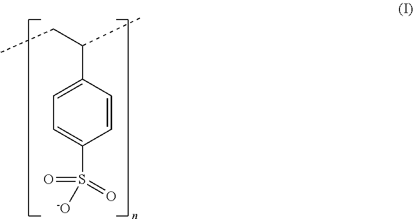

The repeat unit (sulfonate moiety) of the polystyrene sulfonate can be represented by RSO₃⁻, where the sulfonate moiety can exist in equilibrium with hydrogen, sodium, calcium and/or magnesium ions, as shown in Equation (5).

RSO₃⁻H⁺ [symbol] RSO₃⁻Na⁺ [symbol] (RSO₃⁻)₂Ca²⁺ [symbol] (RSO₃⁻)₂Mg²⁺ (5)

Those skilled in the art will understand that the identity of the cation(s) bound to the sulfonate moiety is under thermodynamic control. The type of ions that bind with the sulfonate moiety depends on the binding affinity of the sulfonate moiety for specific ions and the concentration of individual ions in the solution in contact with the polystyrene sulfonate resin. Prior to use for hemodialysis treatment, the polystyrene sulfonate resin is saturated with calcium ions by passing a solution having a high concentration of calcium ions, such as a calcium chloride solution, over the polystyrene sulfonate resin. In certain embodiments, the polystyrene sulfonate resin is provided to the patient or user in a calcium-saturated state. As described herein, in certain embodiments, a polystyrene sulfonate resin is substantially saturated with calcium ions when 50% or more of negatively charged sulfonate moieties present are associated with calcium ions. In other embodiments, a polystyrene sulfonate resin is substantially saturated with calcium ions when 75% or more or 90% or more of negatively charged sulfonate moieties present are associated with calcium ions.

The composition and conductivity for a typical dialysate is shown in Table 1. As shown in Table 1, sodium-containing salts, including NaCl, provide for over 90% of the conductivity of a typical dialysate. An increase in the conductivity of the dialysate over the course of hemodialysis treatment is caused primarily by the addition of sodium ions to the dialysate through the action of the ZrP material.

TABLE 1
Composition and conductivity of a typical dialysate

| Substance | mmol/L | mS/cm |
|---|---|---|
| NaCl | 103 | 10.68 |
| NaHCO₃ | 34.0 | 2.47 |
| KCl | 2.00 | 0.26 |
| CaCl | 1.75 | 0.35 |
| MgCl | 0.50 | 0.09 |
| NaCH₃COO | 3.00 | 0.21 |

| | |
|---|---|
| Total Conductivity 25° C. | 14.05 |

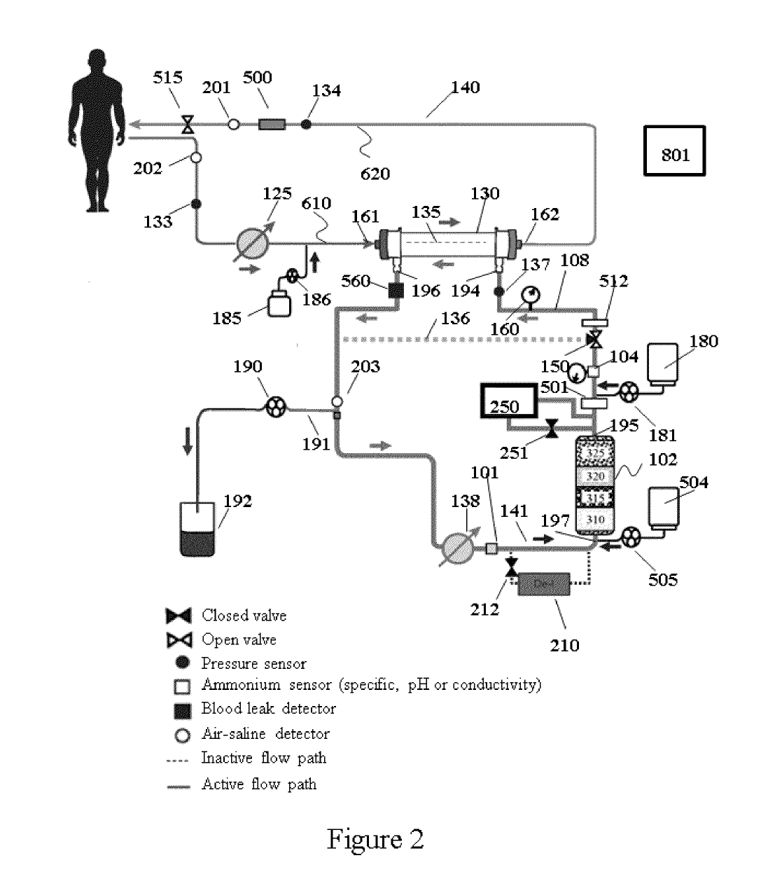

The conductivity of the dialysate is monitored by the one or more conductivity meters 101, 104 and 160 present on the dialysis circuit 141, which are monitored and in communication with one or more controllers 801. As shown in FIG. 1, a polystyrene sulfonate resin cartridge 250 can be incorporated into the hemodialysis system. The polystyrene sulfonate resin cartridge 250 shown in FIG. 2 is connected to the flow path of the dialysis circuit 141 at a location near the outlet 195 of the sorbent cartridge. In the embodiment shown in FIG. 1, the sorbent cartridge 102 contains an activated carbon material 310, a urease-containing material 315, a ZrP material 320 and a ZrO material 325. Control of dialysate flow through the polystyrene sulfonate resin cartridge 250 is controlled by operation of valve 251.

As dialysate leaves the sorbent cartridge 102 through outlet 195, the flow of dialysate through the polystyrene sulfonate resin cartridge 250 (R₁ flow) and the flow of dialysate directly toward the air trap 501 bypassing the polystyrene sulfonate resin cartridge 250 (R₀ flow) and flowing only through the sorbent cartridge 102 is controlled by valve 251 under operation of a controller 801. Several different modalities exist for controlling the R₁/R₀ ratio. In certain embodiments, the actuation of valve 251 is done in response to a conductivity of the dialysate reported by one or more of conductivity meters 101, 104 and 160. When the conductivity of the dialysate increases above a predetermined level, valve 251 is actuated to divert at least part of the dialysate flow exiting the sorbent cartridge 102 through the polystyrene sulfonate resin cartridge 250. Under the control of controller 801, the ratio of R₁/R₀ can be set anywhere from 0 to 1.

Those skilled in the art will understand that only a fraction of the sodium ions passing over the polystyrene sulfonate resin cartridge 250 will be retained. The percentage of sodium ions retained by the polystyrene sulfonate resin cartridge 250 is dependent upon the relative calcium saturation of the polystyrene sulfonate resin and the concentration of the other species in equilibrium with the polystyrene sulfonate resin, as shown in Equation (1), which will vary over the course of treatment. As such, R₁/R₀ can be actively controlled and adjusted over the course of treatment to maintain a stable conductivity.

As described, a trend for increasing conductivity in the dialysate is the result of a stoichiometric exchange of ammonium ions for sodium and hydrogen ions by the ZrP material. As a result, an increase in dialysate conductivity is proportional to the amount of urea being processed by the sorbent cartridge 102. Urea is the main waste species that builds up in the blood of subjects having chronic kidney disease. That is, high urea levels in the blood indicates a combination of poor kidney function to clear waste species and impurity species from the blood and a significant elapse of time from a prior hemodialysis treatment. The blood level of urea in a subject is expected to be correlated with the presence of other waste species that are not being adequately cleared from the blood, such as phosphates and sulfate ions.

As the rate of sodium ions being eluted from the sorbent cartridge 102 increases, the R₁/R₀ is shifted by the controller 801 to pass a greater portion of the dialysate flow through the polystyrene sulfonate resin cartridge 250. As the R₁/R₀ ratio increases, a greater amount of calcium ions are released in exchange for sodium ions. Since the R₁/R₀ ratio is correlated with the overall level of impurity species in the dialysate originating from the patient's blood (e.g. urea, phosphates, sulfate ions, etc.), the amount of calcium ions released inside the polystyrene sulfonate resin cartridge 250 also increases or decreases in response to the overall level of impurity species in the dialysate. The amount of the polystyrene sulfonate resin present in the polystyrene sulfonate resin cartridge 250 can be tailored such that only a low level of calcium ions is eluted from the polystyrene sulfonate resin cartridge 250 during treatment, where substantially all of the calcium ions released by sodium ion exchange are consumed in one of the precipitation reactions outlined in Equations (2)-(4).

In certain embodiments, the polystyrene sulfonate resin cartridge 250 is employed to control the conductivity of the dialysate. Those skilled in the art will understand that only a portion of the sodium ions passing through the polystyrene sulfonate resin cartridge 250 are exchanged for calcium ions. The polystyrene sulfonate resin cartridge 250 operates to reduce the sodium concentration and conductivity in the dialysate to be within a predetermined range. If required, a majority of the dialysate flow can be passed through the polystyrene sulfonate resin cartridge 250 to reduce sodium ion levels to an acceptable range.

The sorbent cartridge 102 functions to remove substantially all Ca²⁺, Mg²⁺ and K⁺ ions from the dialysate. The dialysate entering the polystyrene sulfonate resin cartridge 250 does not contain a substantial quantity of calcium ions. Substantially all of the calcium ions released by the exchange for sodium ions are precipitated and retained with the polystyrene sulfonate resin cartridge 250. The increase in conductivity of the dialysate due to sodium ion released from the sorbent cartridge 102 is proportional to the amount of urea absorbed by the sorbent cartridge 102.

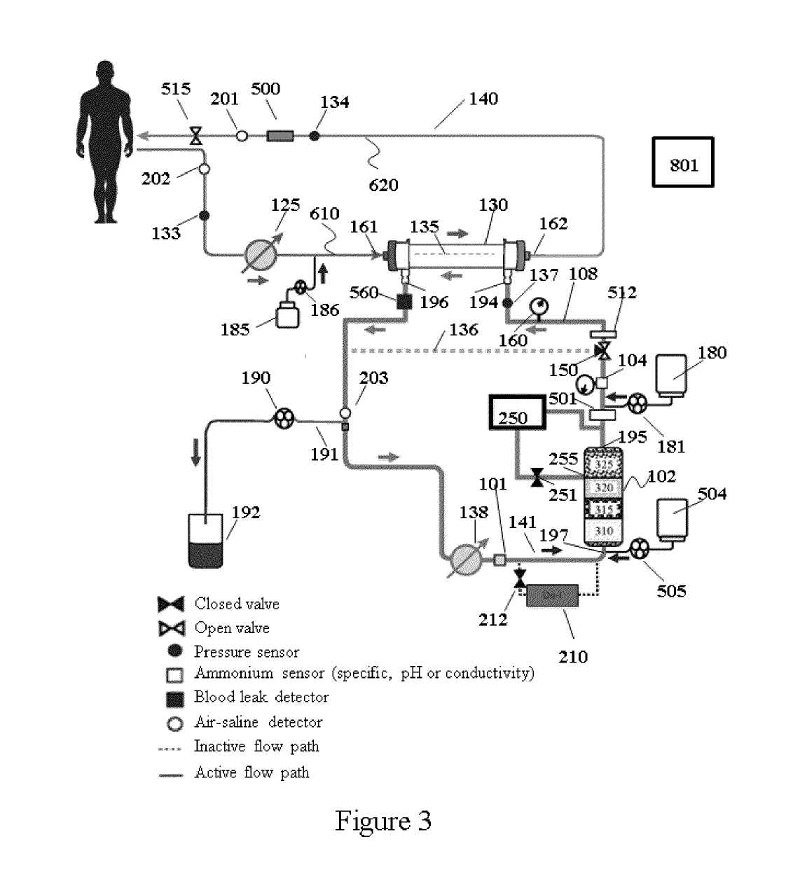

In certain additional embodiments, a deionization cartridge 210 is present in the system for the purpose of generating a diluent to reduce sodium concentration when needed, as shown in FIG. 3. The deionization cartridge 210 contains a mixed bed anion and cation exchange resin, which functions by having a cation exchange resin that releases hydrogen ions in exchange for cations present in the dialysate and an anion exchange resin for exchanging hydroxyl ions for anions present in the dialysate. Dialysate that is passed over the deionization cartridge 210 is substantially deionized and has low conductivity. A portion of the dialysate flow is diverted by means of valve 212 to pass over the deionization cartridge 210. The actuation of valve 212 is controlled in response to the conductivity measured by conductivity meter 101, as shown in FIG. 3, which measures conductivity of the dialysate prior to the location of the deionization cartridge 210. Alternatively, valve 212 can be controlled in response to a conductivity measured by one or more of conductivity meters 104 and 160 to ensure that sodium ion levels and conductivity remains within an acceptable range throughout the dialysis circuit 141. In another embodiment, the sorbent cartridge has two separate flow paths for regenerating a dialysate. The first flow path includes a urease-containing material and a zirconium phosphate material and a second flow path includes a mixed bed anion and cation exchange material.

In the embodiment shown in FIG. 2, the ratio R₁/R₀ for the control of flow through the polystyrene sulfonate resin cartridge 250 is not limited to being controlled in response to the conductivity reported by one or more of conductivity meters 101, 104 and 160. In embodiments where the deionization cartridge 210 is present, diversion of a portion of the dialysate flow through the deionization cartridge 210 can be performed to accurately control sodium ion concentration in the dialysate. Since the diluent eluted from the deionization cartridge 210 is substantially deionized, the controller 801 can accurately determine the need for adjustment of valve 212 to control sodium ion concentration based upon the conductivities reported by conductivity meters 101, 104 and 160.

In embodiments where the deionization cartridge 210 is present, the polystyrene sulfonate resin cartridge 250 is not required to serve as the primary component to remove sodium ions added to the dialysate due to the exchange of ammonium ions in the sorbent cartridge 102. It is understood that the dialysate contains a significant concentration of sodium ions at all times during treatment, where passage of the dialysate through the deionization cartridge 210 will result in exchange of sodium ions for calcium ions. However, due to the availability of the deionization cartridge 210, the ratio R₁/R₀ for the control of flow through the polystyrene sulfonate resin cartridge 250 can be determined by factors other than the conductivity of the dialysate.

For example, the ratio R₁/R₀ for the control of flow through the polystyrene sulfonate resin cartridge 250 can be adjusted based on the impurity load on the sorbent cartridge 102. As will be described below, the amount of urea being removed by the sorbent cartridge 102 can be determined by measuring the conductivity at the inlet 197 and at the outlet 195 of the sorbent cartridge 102. The amount of urea being removed from the patient is expected to be correlated with the level of other impurities passing from the blood in the extracorporeal circuit 140 to the dialysis circuit 141 via the dialyzer 130. A patient having poor kidney function leading to a high level of urea build-up in the blood will likely have high levels of other species that are normally kept in a tight homeostatic range by the kidneys by those having normal kidney function, including sulfate ions and phosphates.

At the beginning of treatment, the level of urea and other impurities in the patient's blood is at the highest point during the course of treatment. As treatment progresses, the level of impurity species and waste products in the patient's blood, and hence transported by the dialysate from the dialyzer 130, decreases over the course of treatment. The ratio R₁/R₀ for the control of flow through the polystyrene sulfonate resin cartridge 250 can be adjusted during the course of treatment in response to the overall level of impurities expected in the dialysate based upon the amount of urea being removed by the sorbent cartridge 102.

In additional embodiments, the ratio R₁/R₀ for the control of flow through the polystyrene sulfonate resin can be controlled in response to the amount of calcium ions observed to be eluted from the polystyrene sulfonate resin cartridge 250. A calcium sensing electrode can be located at a position after the polystyrene sulfonate resin cartridge 250 in the dialysis circuit 141. For example, the calcium sensing electrode can replace conductivity detector 104 or be co-located with conductivity detector 104. As the dialysate is circulated through the dialysis circuit 141 and passed through the sorbent cartridge 102, the sorbent cartridge 102 removes the non-sodium cations from the dialysate (e.g. Mg²⁺, Ca²⁺, K⁺, etc.) through action of the ZrP material. The dialysate eluting from the outlet 195 of the sorbent cartridge 102 and diverted through the polystyrene sulfonate resin cartridge 250 is substantially depleted in calcium ions. Under normal operating conditions, necessary cations are reinfused into the dialysate using an infusate solution provided in container 180 using infusate pump 181. The point at which the infusate (i.e. cation) solution is added to the dialysate can be between the sorbent cartridge 102 and the valve 150 in any embodiment or between the sorbent cartridge 102 and the dialysate inlet 194 of the dialyzer 130 in other embodiments. In any embodiment, the infusate solution provided in container 180 can be lacking a calcium salt to prevent interference with the calcium sensing electrode.

As shown in FIGS. 1 and 2, the polystyrene sulfonate resin cartridge 250 is placed on the dialysis circuit 141 at a position between the outlet 195 of the sorbent cartridge 102 and the point at which infusate is introduced to the dialysis circuit 141 by the infusate pump 181. The calcium sensing electrode is able to monitor calcium ions originating from the polystyrene sulfonate resin cartridge 250 if placed at a location after the polystyrene sulfonate resin cartridge 250. The ratio R₁/R₀ can be optimized to balance the exchange of sodium ions for calcium ions shown in Equation 1 with the precipitation reactions shown in Equations (2)-(4). When the ratio R₁/R₀ is too high, not all of the exchanged calcium ions will participate in one of the precipitation reactions, Equations (2)-(4), and calcium will escape from the polystyrene sulfonate resin cartridge 250 and be detected by the calcium sensing electrode. In any embodiment, the ratio R₁/R₀ is adjusted by the controller 801 such that only a minimal amount of calcium ions are detected by the calcium sensing electrode. When a minimal amount of calcium ions is detected, the ratio R₁/R₀ is at a level where a sufficient amount of calcium ions are released by exchange with sodium ions to achieve the maximum amount of precipitation of carbonate ions, sulfate ions and phosphates. When no calcium ions are detected by the calcium electrode, the amount of calcium ions being released by the polystyrene sulfonate resin is likely not sufficient to achieve the maximum amount of precipitation possible, which indicates a need to increase the ratio R₁/R₀.

In certain embodiments, the ratio R₁/R₀ can be adjusted such that calcium ions are eluted from the polystyrene sulfonate resin cartridge 250 at a targeted rate. As described, cations, including calcium ions, are removed from the dialysate by sorbent cartridge 102 requiring a constant replacement of the removed cations. The ratio R₁/R₀ can be adjusted such that the replacement calcium ions are provided by the polystyrene sulfonate resin cartridge 250. The controller 801 can quantify the amount of calcium being emitted by the polystyrene sulfonate resin cartridge 250 through application of the Nernst equation and adjust the ratio R₁/R₀ to provide a sufficient amount of replacement calcium ions. A source for calcium ions can be omitted from the infusate solution in container 180.

### Placement of the Polystyrene Sulfonate Resin

As shown in FIGS. 1 and 2, one possible location for the diversion of dialysate though the polystyrene sulfonate resin cartridge 250 is at the outlet 195 of the sorbent cartridge 102. The sorbent cartridge 102 shown in FIGS. 1 and 2 has an activated carbon material layer 310, a urease-containing layer 315, a zirconium phosphate material layer 320 and a zirconium oxide material layer 325 in that order from the inlet 197 to the outlet 195 of the sorbent cartridge 102. Those skilled in the art will readily understand that the order of the materials within the sorbent cartridge 102 can be modified. When the polystyrene sulfonate resin cartridge 250 is present at the outlet 195 of the sorbent cartridge 102, the main ions present that can be precipitated by the polystyrene sulfonate resin cartridge 250 are sulfate ions and carbonate ions. Neither sulfate ions nor carbonate ions are actively absorbed by the materials of the sorbent cartridge 102. During most treatment regimens, it is expected that the concentration of sulfate ions will be higher than carbonate ions.

An alternative placement of the polystyrene sulfonate resin cartridge 250 is through a side port 255 placed in the sorbent cartridge 102, as shown in FIG. 3. All or a portion of the dialysate exiting zirconium phosphate material layer 320 can be diverted through the polystyrene sulfonate resin cartridge 250 as described above. The dialysate diverted through the polystyrene sulfonate resin cartridge 250 in the arrangement shown in FIG. 3 has not been contacted with the zirconium oxide material 325. As such, significant amounts of phosphate are present that can be precipitated by the polystyrene sulfonate resin cartridge 250. Significant amounts of sulfate ions are present as well. In the arrangement shown in FIG. 3, Equations (3) and (4) are the dominate precipitation reactions to occurs within the polystyrene sulfonate resin cartridge 250. Carbonate ions are not expected to be a principal species involved in precipitation due to the generation of hydrogen ions by the activity of the zirconium phosphate material layer 320. A by-product of the conversion of urea to ammonium ions by the urease-containing material is carbon dioxide. The carbon dioxide is removed by air trap 501, which is placed near the outlet 195 of the sorbent cartridge 102, as shown in FIG. 1.

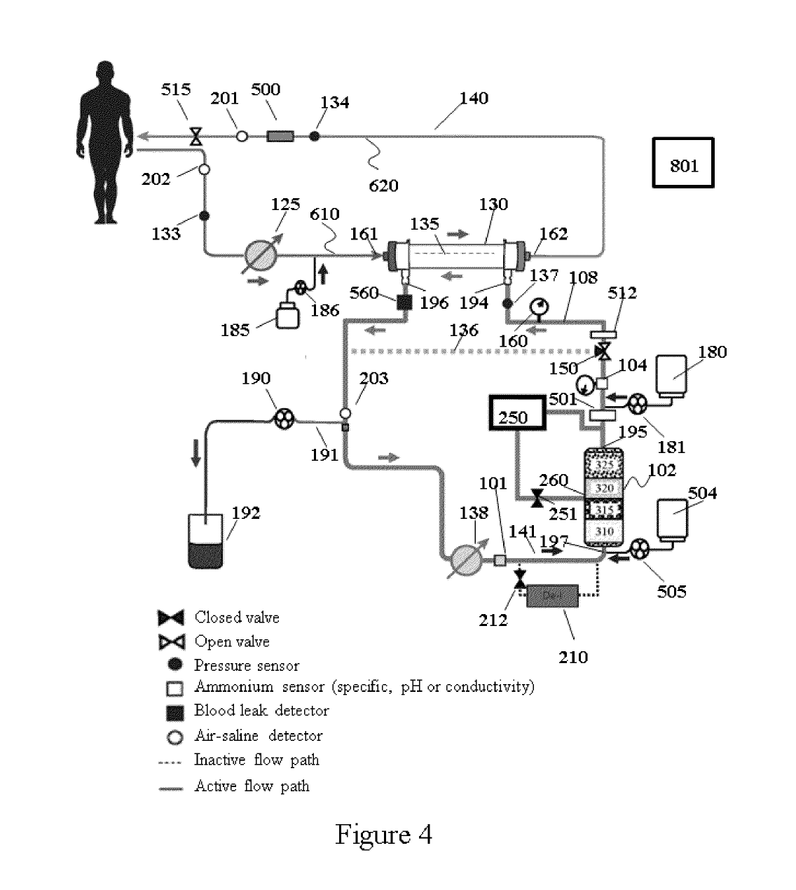

A further alternative placement of the polystyrene sulfonate resin cartridge 250 is through a side port 260 placed in the sorbent cartridge 102, as shown in FIG. 4. All or a portion of the dialysate exiting the urease-containing material layer 315 can be diverted through the polystyrene sulfonate resin cartridge 250 as described above. The dialysate diverted through the polystyrene sulfonate resin cartridge 250 in the arrangement shown in FIG. 4 has not been contacted with the zirconium phosphate material 320. As such, significant amounts of phosphates, sulfate ions and carbonate ions are present that can be precipitated by the polystyrene sulfonate resin cartridge 250. In the arrangement shown in FIG. 4, Equations (2), (3) and (4) are all precipitation reactions that are expect to occur to a significant degree within the polystyrene sulfonate resin cartridge 250.

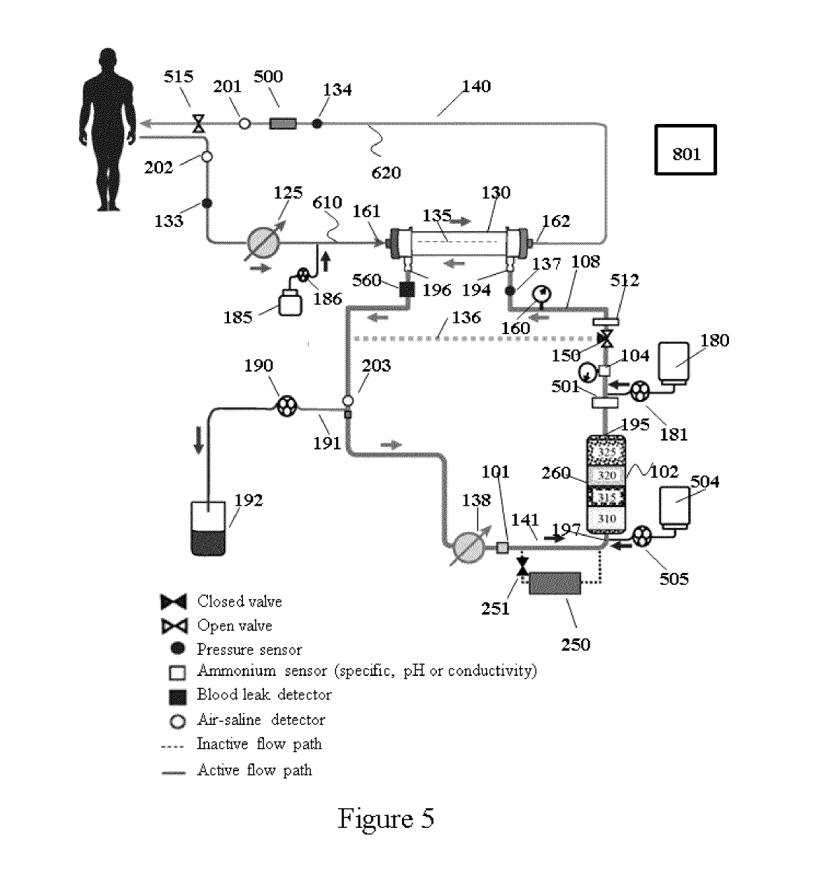

A further alternative placement of the polystyrene sulfonate resin cartridge 250 is at a location between the dialyzer 130 and the inlet 197 of the sorbent cartridge 102, as shown in FIG. 5. As shown in FIG. 5, dialysate is diverted through the polystyrene sulfonate resin cartridge 250 before reaching the sorbent cartridge 102. As such, any phosphates, sulfate ions and carbonate ions that diffused into the dialysate at the dialyzer 130 are present in the dialysate when contacted with exchange resin within the polystyrene sulfonate resin cartridge 250. Therefore, equations (2), (3) and (4) are all precipitation reactions that are expect to occur to a significant degree within the polystyrene sulfonate resin cartridge 250. The ratio R₁/R₀ for the fraction of dialysate passing through the polystyrene sulfonate resin cartridge 250 can be determined using any of the considerations described above. However, any calcium ions that may elute from the housing of the polystyrene sulfonate resin cartridge 250 will be absorbed by the sorbent cartridge 102 in the configuration displayed in FIG. 5.

### Control of pH

Constant pH in the blood is maintained by the presence of bicarbonate which is equilibrated with CO₂ through the action of carbonic anhydrase. Systems employing a sorbent cartridge 102 have a tendency to induce mild acidosis, particularly toward the beginning of treatment, due to adsorption of bicarbonate by the sorbent cartridge 102, where bicarbonate freely diffuses across the dialysis membrane. After the initial loss of bicarbonate to the sorbent cartridge, the sorbent cartridge will add bicarbonate to the dialysate due to hydrogen ions added to the dialysate in exchange for ammonium carbonate.

In certain other embodiments, a bicarbonate containing solution can be added via a bicarbonate pump 505, as shown in FIGS. 1-5. A bag or container 504 containing sodium bicarbonate is attached to the system via a pump 505. The bag or container 504 can contain a premeasured solid amount of sodium bicarbonate that can be reconstituted during a priming process by running the pump 505 in reverse. Alternatively, a premade bicarbonate solution can be provided. Bicarbonate infusion is controlled by the pump 505 to maintain a constant and physiological pH in the system and to maintain the concentration of bicarbonate ion within predetermined ranges. In other embodiments, the level of bicarbonate ions can be determined by a change in conductivity since bicarbonate salts are conductive. Those skilled in the art will readily understand that the bicarbonate pump 505 and the container 504 can be located at other positions in the dialysis circuit 141.

Bicarbonate ions and carbonic acid are readily exchanged across the dialysis membrane 135. The main sources for acidosis during the treatment process are 1) the ability of the sorbent cartridge to initially absorb bicarbonate ions (lowers pH); 2) reaction of ammonia generated by urease to form ammonium ions (raises pH) 3) generation of hydrogen ions by exchange with ammonium ions; and 4) reaction of CO₂ generated by urease with water to form carbonic acid (lowers pH). The pH of the bicarbonate buffer system present in the dialysate is a function of the ratio of carbonic acid to bicarbonate, where an excess in carbonic acid (dissolved CO₂) causes acidosis. As described above, the polystyrene sulfonate resin cartridge 250 serves to precipitate CO₂ formed by the action of urease thereby removing a principal source of pH change during hemodialysis treatment. The generation of ammonium ions is largely offset by the release of hydrogen ions upon cation exchange with the ZrP material. As such, the use of the polystyrene sulfonate resin cartridge 250 helps stabilize the pH of the dialysate during hemodialysis treatment.

However, bicarbonate ions can be added to the dialysis circuit 141 by the bicarbonate pump 505 as needed or at a constant rate to prevent acidosis. As will be described below, the systems described herein can have an ability to quantify the amount of urea absorbed by the sorbent cartridge, which can then direct the rate of bicarbonate addition. Alternatively, a pH meter (not shown) can be located at a position along the dialysis circuit to monitor the dialysate pH.

### Quantization of Urea Removal

The blood of patients undergoing a regime of renal replacement therapy typically undergoes blood chemistry determination by laboratory testing on a periodic basis to determine the effectiveness of treatment. Such testing is undertaken by a trained healthcare professional on a separate basis from the renal replacement therapy. Based upon lab results, various treatment metrics can be adjusted. For a patient utilizing the wearable sorbent system described herein without the aid of a healthcare professional, it is desirable to have a facility to determine the accuracy of treatment during therapy.

During treatment, the sorbent cartridge acts as a cation exchanger and releases hydrogen and sodium ions. The release of sodium by the sorbent cartridge has two principal sources:

1) Urea is converted to ammonium carbonate by the urease layer of the sorbent cartridge. The ammonium carbonate is exchanged to sodium and hydrogen in the zirconium phosphate layer(s) of the sorbent cartridge. The stoichiometry of the amount of sodium given off in this exchange is dependent on the processing of the zirconium phosphate layer; however, each process provides uniform results. Once the stoichiometry of ammonium/hydrogen/sodium exchange is known, the amount of sodium released from the sorbent cartridge can be used to quantify the amount of ammonium ions absorbed. By means of example, a representative example of the zirconium phosphate material can operate to exchange 1 mEq ammonium ion for 0.15 mEq sodium ion and 0.85 mEq hydrogen ion. In this example, if the cartridge removes 20 grams of urea during a treatment, then the zirconium phosphate material removes 1400 mEq ammonium ions, which would produce about 210 mEq of sodium ions. Those skilled in the art will readily recognize that other zirconium phosphate materials having a different stoichiometry of ammonium/hydrogen/sodium exchange can also be used to calculate the amount of urea converted to ammonium ion and absorbed by the sorbent cartridge; and
2) The dialysis solution contains electrolytes such as calcium (Ca²⁺), magnesium (Mg²⁺) and potassium (K⁺). These electrolytes remain in a stable range and close to constant in the dialysate during treatment. These electrolytes are totally removed from the spent dialysate by the sorbent cartridge. To ensure that there is a stable and correct concentration of electrolytes in the refreshed dialysate prior to reaching the dialyzer, zirconium phosphate exchanges these electrolytes with sodium ions. Then, the electrolytes are re-infused via an infusate pump to the correct concentrations. The amount of sodium produced from the zirconium phosphate layer due to this exchange is dependent on the dialysis solution flow rate, the time of treatment and the concentration values of these cations in the dialysis solution. For example, if the Ca²⁺ were 3 mEq, the Mg²⁺ 1 mEq, and the K⁺ 1 mEq, the sorbent cartridge would produce approximately 240 mEq of sodium at a 20 ml/min flow rate and a total volume of 48 liters through the cartridge.

Due to the near constant amounts of (Ca²⁺), magnesium (Mg²⁺) and potassium (K⁺) ions being exchanged by the sorbent cartridge, the conductivity difference between dialysate containing urea entering the sorbent cartridge compared with the refreshed dialysate exiting the sorbent cartridge can be used to quantify the amount of urea converted to ammonium ions and absorbed by the sorbent cartridge. As described above, the concentration of calcium ions in the dialysate can undergo some variation in concentration over the course of treatment depending upon the ratio R₁/R₀ of dialysate flow over the polystyrene sulfonate resin cartridge 250. However, as shown in Table 1, calcium ions in the form of calcium chloride are a minor contributor to the overall conductivity of the dialysate contributing about 2% of expected conductivity. Minor fluctuations in the concentration of calcium ions are not expected to significantly affect the ability to quantify the absorption of urea by the sorbent cartridge 102. If the temperature and composition of an electrolyte solution are constant, the resulting conductivity of the solution will remain stable. At the ranges of typical dialysis solutions, any change in sodium concentration will result in a linear increase or decrease in dialysate conductivity. Table 1 above shows the concentration and conductivity of a typical dialysis solution at 25° C. Even though sodium is not the only contributor to conductivity in dialysis solution, NaCl and NaHCO₃ make up approximately 94% of the conductivity of a typical dialysate solution.

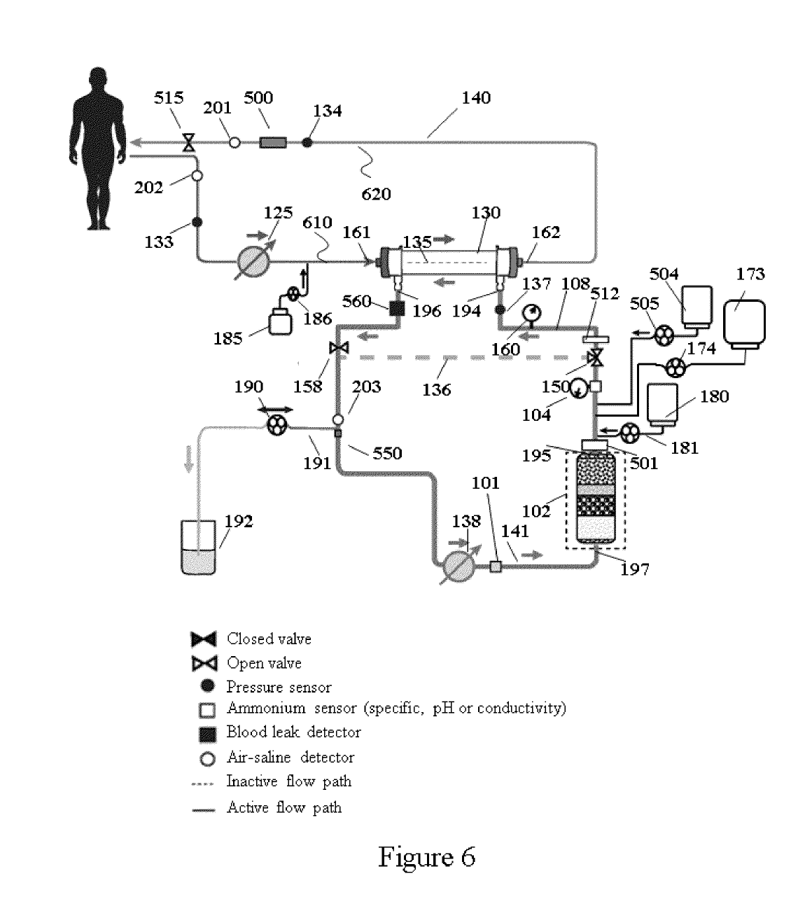

Sodium concentration increases in the dialysate due to the exchange of ammonium to sodium, which can be used to verify if the urea was removed during the course of treatment. As shown in FIG. 6, conductivity meters 101, 104 and 160 can be incorporated into the system to measure the conductivity of dialysate traveling to the inlet 197 and exiting the outlet 195 of the sorbent cartridge 102. For simplicity, the deionization cartridge 210 and the polystyrene sulfonate resin cartridge 250 are not shown in FIG. 6. In certain embodiments, a conductivity meter can be present within the sorbent cartridge at the outlet of the zirconium phosphate layer 320. A microprocessor or controller 801 can monitor the conductivity measured by the conductivity meters to analyze the changes in conductivity brought about by the following:
1) Conversion of urease to ammonium carbonate and subsequent exchange of ammonium carbonate to sodium, and
2) Any net change in conductivity due to the exchange of Ca²⁺, Mg²⁺, and K⁺ into sodium, which can be treated as a constant value. The change due to removal of Ca²⁺, Mg²⁺, and K⁺ is known and the increase due to sodium is known. In the example dialysis solution of Table 1, the Ca²⁺, Mg²⁺, and K⁺ contribute 0.7 mS/cm of conductivity.

The change in conductivity due to the loss of Ca²⁺, Mg²⁺, and K⁺ and the increase of sodium ions due to this exchange will be relatively constant during the treatment. From this information, a controller can then calculate the amount of conductivity increase due to the urea removal via the following sources:

Inlet Conductivity−Conductivity Contribution of Ca²⁺, Mg²⁺, and K⁺=Starting Conductivity

Outlet Conductivity−Increase in Conductivity due to exchange of Ca²⁺, Mg²⁺, and K⁺ to Na⁺=Corrected Outlet Conductivity

Corrected Outlet Conductivity−Starting Conductivity=Conductivity Increase due to Conversion of NH₄⁺ to Na⁺

The following example quantization is based upon 48 liters of regenerated dialysis solution used during the course of treatment having typical concentrations of Ca²⁺, Mg²⁺, and K⁺:

Inlet Conductivity=14.04 mS/cm Outlet Conductivity=14.32 mS/cm
1. 14.05 mS/cm−0.7 mS/cm=13.35 mS/cm Starting Conductivity
2. 14.32 mS/cm−0.5 mS/cm=13.8 mS/cm Corrected Outlet Conductivity
3. 13.8 mS/cm−13.35 mS/cm=0.45 mS/cm Conductivity Increase due to Conversion of NH₄⁺ to Na⁺
4. 0.45 mS/cm/0.1037 mS·L/mEq·cm=4.34 mEq/L Na⁺ due to Urea Removal
5. 0.4 g urea per liter

In hemodialysis, urea removal depends on the diffusive gradient across the dialyzer membrane. This gradient will be much higher at the beginning of treatment than at the end of treatment when typically 50 to 60 percent of the patient's urea has been removed. In certain embodiments, the conductivity values can be averaged so the curve of urea removal is understood and a continuous calculation need not be made. For example, conductivity can be sampled four or five times per treatment session for the purposes of quantifying urea removal. Early during a treatment session, a quantization of urea removal can be performed to verify that urea is being removed and that the Na⁺ increase is relatively high. Later, quantization measurements can be performed to calculate a curve for urea removal and to predict total expected urea removal based on this curve. The amount of urea removed during treatment can be either accurately measured or estimated with a high degree of certainty.

### Detection of Significant Clearance Problems

The urea removal monitoring facility described above can be used to indicate the proper operation of the system and to alert the patient to significant problems that would interrupt the waste removal process. This problem could be communicated automatically via WiFi, the internet, or other communication means to the doctor or healthcare professional. For instance a patient with impaired blood access flow would have little urea removed. In instances were low urea removal is monitored toward the beginning of treatment, an alarm can be communicated indicating a potential malfunction.

Access to the patient's vasculature can fail due to a buildup of plaque in the access stent. This plaque creates a stenosis at the distal end of the anastomosis where the stent or graft is sutured to the vascular system of the patient. When this occurs, the blood tends to recirculate within the access area and there is a lack of adequate flow of fresh blood into the extracorporeal circuit, which can result in the same blood being repeatedly dialyzed. Since little blood entering the dialyzer is from the systemic circulation, there is less urea in the blood and hence less sodium is produced from the cartridge due to urea/ammonium to sodium exchange. The lack of an adequate increase in conductivity can be detected by the system and an alert can be sent indicating a potential malfunction or problem accessing the patient's vascular system. This alert can indicate a lowered waste clearance, but the alert does not necessarily specify if the cause of the lowered waste clearance is due to a vascular access problem or due to a problem in dialysis flow, etc. A skilled medical professional can analyze the event to determine the cause of the alert in some embodiments.

### Detection of Zirconium Exhaustion

After an extended period of use, the ability of the zirconium phosphate to absorb urea can be exhausted. Exhaustion of zirconium phosphate leads to ammonium release into the dialysate, which can lead to ammonium intoxication in the patient. As discussed above, the exchange of urea/ammonium to sodium affects the output conductivity of the sorbent cartridge. Monitoring the inlet and outlet conductivities of the cartridge thus provides a method to detect ammonium breakthrough in the sorbent cartridge. An equilibration of the sorbent cartridge inlet conductivity with the output conductivity over a short time period indicates that the zirconium phosphate layer within the sorbent cartridge is exhausted. In certain embodiments, the conductivities pre- and post-sorbent cartridge is monitored. If an increase in sodium concentration is not detected by the controller, then the system will send an alert and prevent the dialysate from reaching the dialyzer, thus protecting the patient from ammonia intoxication.

### Ultrafiltration (UF) and Increased Convective Clearance

The compliance control pump 190 is operated by a controller 801 that accurately accounts for the volume of fluid being removed from the circulation of the patient and/or being infused into the circulation of the patient. As described above, the compliance control pump 190 controls the movement of fluid across the dialysis membrane 135 due to the controlled compliant volume of dialysis circuit 141. Many kidney failure patients can have fluid build-up that may be addressed by ultrafiltration, where bulk fluid is removed via the circulatory system. The compliance control pump 190 can be accurately used to determine the precise volume of fluid removed from the patient. In addition to accurately controlling the net fluid removed and the convective clearance of a patient, accurate control of the efflux or influx of fluid via the compliance control pump 190 allows for the amount of sodium removed (mEq Na⁺) during a course of treatment to be determined, where such result can be calculated and stored in the memory of a controller 801 and/or be displayed on a control panel (not shown). Accurate control of bulk fluid movement across the dialysis membrane can further be used to enhance clearance of mid-weight impurities by convective clearance, which is particularly beneficial for mid-weight impurities such as β-2 microglobin which are not removed very well by hemodialysis and which higher serum blood levels are associated with higher patient mortality. To be able to control net patient fluid removal, any fluid removed in excess of the desired patient fluid loss must be reinfused to the blood. This is accomplished in one embodiment by running the control pump 190 in reverse during the treatment and then compensating by ultrafiltration: Control Pump Control=Net patient UF+Convective UF. Control pump backfiltration is controlled to Convective UF volume. For example, a desired 200 ml net patient fluid loss per hour and 1000 ml of convection per hour requires a control pump 190 running at a UF rate (efflux rate) of 1000 ml/hr and at a backfiltration rate (influx rate) of 800 ml/hr to achieve the net fluid loss and the desired convective clearance. These same mechanisms allow one to give fluid to the patient when necessary, rinse back blood and control fluid removal accurately.

The rate of diffusion of a solute is dependent upon the molecular weight of that solute. Small molecules, such as urea, can effectively diffuse from the extracorporeal side of the dialysis membrane to the dialysate side of the dialysis membrane in the absence of net movement of fluid. However, larger, mid-sized molecules, having a lower rate of diffusion may not be removed as effectively. As used herein, the term mid-sized molecule refers to an impurity having a molecular weight greater than about 1000 g/mol and less than about 66000 g/mol includes uremic toxins, B12, C reactive protein, and β2-microglobin.

During periods of net movement of fluid from the extracorporeal side to the dialysate side of the dialysis membrane 135, solutes can be dragged across the dialysis membrane 135 along with the net movement of fluid. This process, referred to as convective clearance, removes mid-weight impurities from the patient's blood, which are absorbed by the sorbent cartridge 102. Some convective clearance occurs during the course of ultrafiltration as described above. However, the amount of convective clearance is limited by the volume of fluid that is removed by ultrafiltration. For example, if 1 L of fluid is to be removed from the patient over the course of a 4-hour treatment, then the amount of convective clearance that occurs due to 1 L of fluid crossing the dialysis membrane 135 is the maximum amount of convective clearance that occurs during the treatment regimen. Without infusing the patient with additional fluid, the amount of fluid that can be removed is limited considering that the average individual has about 5 L of blood. Further, it may be desirable to achieve convective clearance without the removal of a large amount of fluid from the patient.

To achieve convective clearance in accordance with certain embodiments, the control pump 190 is operated in the efflux direction to pull fluid from the extracorporeal circuit 140, and hence from the patient, across the dialysis membrane 135. During the net efflux for fluid across the membrane 135, mid-weight solutes and impurities are carried into the circulating dialysate where they can be absorbed by the sorbent cartridge 102. The control pump 190 is periodically reversed to the influx direction to force fluid from the control reservoir 192 into the controlled compliance dialysis circuit 141 and thereby force a corresponding volume of fluid into the extracorporeal circuit 140 and into the patient.

Under a regime where the control pump 190 is run in the efflux and influx directions for approximately equal amounts of time at the same pump rate, the amount of convective clearance will be approximately the efflux flow rate without causing any net addition or removal of fluid from the patient. For example, if the compliance control pump 190 is run at 10 mL/min for a hour with periodic reversal between efflux and influx directions, then 300 mL of fluid is moved from the extracorporeal circuit into the controlled compliance dialysis circuit 141 to affect convective clearance, where the same volume is returned to the patient resulting in no net fluid removal at the end of treatment. In the alternative, the time that the compliance control pump 190 is operated in the efflux or influx direction can be unequal to affect a net volume of ultrafiltration during the course of treatment. For example, if the control pump 190 is operated in the efflux direction for 18-second periods with intervening 12-second periods in the influx direction at a rate of 10 mL/min, then 360 mL/h of fluid is moved in the efflux direction to affect convective clearance and a net of 120 mL/h of fluid is removed from the patient. Those skilled in the art will understand that the interval at which the control pump 190 operates between efflux and influx directions can be modified to further effect the amount of convective clearance and net ultrafiltration occurring over the course of treatment.

The blood pump 125 and the dialysate pump 138 provide the majority of the energy to convey the blood through the extracorporeal circuit 140 and the dialysate through the controlled compliance dialysis circuit 141, respectively. In certain embodiment, the blood pump and the dialysate pump can be independently operated at any rate in a range from about 50 to about 300 mL/min including from about 60 to about 295 mL/min is contemplated by the invention such as about 76 to about 185 mL/min, about 85 to about 287 mL/min, about 25 to about 115 mL/min, about 45 to about 273 mL/min, about 156 to about 293 mL/min, about 32 to about 163 mL/min, about 145 to about 199 mL/min, about 167 to about 193 mL/min or, about 29 to about 224 mL/min. In certain embodiments, the blood pump and/or the dialysate pump deliver a constant load pressure such that the conveyance rate is constant over at least short periods of times. Pumps that can deliver a constant load pressure include peristaltic pumps.

The use of pulsatile pumps, that mimic the pulsing action of the human heart, has been proposed to enable convective clearance. As discussed herein, in known devices, the blood and the dialysate are conveyed by pulsatile pumps that are set 180 degree out of phase in order to achieve periodic filtering across the dialysis membrane. When the blood pump is undergoing a pulse action and the dialysate pump is at rest, convective clearance can occur due to an increase in pressure difference across the dialysis membrane. Conversely, fluid is back filtered across the dialysis membrane when the dialysate pump is undergoing a pulse action and the blood pump is at rest. However, such systems have been subject to increased clotting. It is desirable to stop the administration of heparin or other anticoagulant 30 to 60 minutes prior to the end of dialysis to restore normal clotting by the time treatment ends. However, blood becomes significantly more viscous at low flow rates. In addition, protein coats the membrane surface starting the clotting cascade. The periodic slow down of blood circulation caused by the action of a pulsatile pump contributes to clotting occurring in the extracorporeal circuit. Blood clotting prevents the completion of treatment.

The above-described method for performing convective clearance using pulsatile pumps requires the flow rate of the blood and the dialysate through the dialyzer to be similar to function properly. The pressure generated in the dialyzer on either side of the dialysis membrane is dependent upon the flow rate, where the flow rate of the dialysate and the blood should be close to achieve equal movements of fluid in both directions across the dialysis membrane. Specifically, the ratio of blood flow to dialysis flow has been recommended to be from 3:4 to 4:3 when employing pulsatile pumps to increase convective clearance. The use of pulsatile pumps to perform convective clearance also increases hemoconcentration, which increases the risk for blood clotting. As the flow rate of blood through a dialyzer is lowered relative to the flow rate of dialysate through the dialyzer, any particular volume of fluid pulled from the extracorporeal circuit during a unit value of time causes a greater amount of hemoconcentration. The volume of fluid removed from the extracorporeal circuit is removed from a smaller volume of blood as the flow rate of blood is lowered. As described above, a ratio of blood flow to dialysis flow has been recommended to be from 3:4 to 4:3 when pulsatile pumps are used to create convective clearance. Using the controlled compliance dialysis circuit described herein, the net flux of fluid across the dialysis membrane 135 is controlled by the control pump 190 rather than a ratio of flow rates between blood and dialysate. The ratio of blood flow to dialysate flow can be set at a value that reduces hemoconcentration as a result of pulling fluid from the extracorporeal circuit. In certain embodiments, the ratio of blood flow to dialysate flow through the dialyzer 130 is from about 1:1.5 to 3:1, and can include any range of ratios in between. In certain additional embodiments, the ratio of blood flow to dialysate flow through the dialyzer 130 is from about 1.5:1 to 3:1, and can include any range of ratios in between. In certain other embodiments, the rate of blood flow through the dialyzer 130 is at least about 50% greater than the rate of dialysate flow through the dialyzer 130.

As shown in FIG. 6, a second control reservoir 173 and second reservoir pump 174 can be present in any embodiment of the systems described herein. For simplicity of presentation, polystyrene sulfonate resin cartridge 250 is not shown in FIG. 6; however, those skilled in the art will understand that polystyrene sulfonate resin cartridge 250 can be present in any position as shown in FIGS. 1-5 along with the second control reservoir 173 and second reservoir pump 174. While the second control reservoir 173 and second reservoir pump 174 are shown connected to the dialysis circuit 141 at a specific location in FIG. 6, those skilled in the art will recognize that the second control reservoir 173 and second reservoir pump 174 can be integrated into any embodiment described herein and the location of attachment to the dialysis circuit 141 can be modified. As described above, the control pump 190 can be operated in a bidirectional fashion to assist in the performance of convective clearance. Specifically, the control pump can be operated in the efflux direction to cause the movement of fluid from the extracorporeal circuit 140 into the dialysis circuit 141 and in the influx direction to cause the movement of fluid from the dialysis circuit 141 into the extracorporeal circuit 140.

In certain embodiments, operation of the control pump 190 in the influx direction can be substituted with operation of the second reservoir pump 174 to drive liquid from the second control reservoir 173 into the dialysis circuit 141 and subsequently cause movement of fluid from the dialysis circuit 141 into the extracorporeal circuit 140 across the dialysis membrane 135. The control pump 190 can be used for the movement of fluid in the opposite direction across the dialysis membrane 135. The second reservoir pump 174 and second reservoir 173 are used for the performance of convective clearance in embodiments of the invention where the total void volume of the dialysis circuit and working dialysate is less than about 0.5 L, or in embodiments where the void volume of the dialysis circuit and working dialysate is less than 1 L.

The second control reservoir 173 can hold water, tap water or purified water. As discussed, the exchange of ammonium ions by the sorbent cartridge 102 has a tendency to release sodium ions into the dialysate. The second control reservoir 173 can have the same structure as control reservoir 192 including the presence of an air filter similar to air filter 300. While the concentration of sodium ions in the dialysate can be reduced through the operation of the control pump 190 in the efflux direction, diversion of the dialysate through the deionization cartridge 210 or polystyrene sulfonate resin cartridge 250, these routes to reduction sodium ion concentration in the dialysate may potentially not be sufficient to reduce sodium ion concentration. As such, the second reservoir pump 174 can be operated to add water or another fluid as a diluent to the dialysis circuit 141 to reduce sodium concentration as needed.

In certain embodiments, the volume of fluid held by the second control reservoir 173 is about 1 L or less, or about 0.5 L or less. In certain embodiments, the volume of the fluid held by the reservoir is from about 0.1 to about 1 L, from about 0.2 to about 0.8 L, from about 0.5 to about 1 L, from about 0.6 to about 1 L, from about 0.5 to about 0.8 L or from about 0.2 to about 0.8 L.

### Detection of Patient Hydration Status

The portable dialysis described herein can be used to perform ultrafiltration on a patient. During ultrafiltration, fluid is drawn out from the serum of the blood in the extracorporeal circuit through the dialysis membrane 135 by means of the control pump 190. Fluid removed by the control pump 190 is removed to the control reservoir 192. Ultrafiltration can be performed alone or in conjunction with convective clearance, as described above.

Patients having kidney failure may have an undesirable accumulation of fluid in body tissues that is called edema. As fluid (e.g. water) is removed from the patient's plasma, the volume of the patient's plasma is replaced by infusion of fluid from the patient's tissues. That is, the portable dialysis system does not directly access fluids stored in the patient generally but only directly accesses the patient's vascular system. Humans typically only have 5 to 6 L of plasma volume at any one time, where a significant time lapse can be required for plasma volume to be replaced by transfer of fluid from surrounding tissues.

During ultrafiltration, fluid can be removed too rapidly resulting in the patient becoming hypovolemic, which can cause several serious effects including hypotension, cramping, nausea and vomiting. To avoid instances of hemoconcentration due to excessive fluid removal, the rate of ultrafiltration is limited to a percentage of the blood flow through the extracorporeal circuit 140. In certain embodiments, the rate of ultrafiltration is limited to be no greater than about 30% of the plasma flow through the extracorporeal circuit 140. Plasma flow (Qp) is defined as Qp=Blood flow rate x (1-hematocrit), where blood flow rate is in units of volume divided by time (e.g. mL/min) and hematocrit is the unitless fraction of blood volume occupied by red blood cells. For example, if the blood flow rate is 60 mL/min and the hematocrit is 40%, then the maximum rate of ultrafiltration is set to be equal to about 10.8 mL/min or less.

The portable dialysis system can have a hematocrit detector to determine the hematocrit of blood containing within the extracorporeal circuit 140. In certain embodiments, the hematocrit detector is a light source and a photodetector, wherein light emanating from the light source is passed through the blood in the extracorporeal circuit 140 and detected by the photodetector. The absorption of one or more wavelengths of light can indicate the level of hematocrit in blood entering the dialyzer 130 in the arterial line 610. In certain embodiments, the hematocrit detector gives an indication if the hematocrit trend is unsafe rather than giving a precise numerical quantification. In certain additional embodiments, the hematocrit detector can also determine if blood is present in the extracorporeal circuit 140, which can be useful during the processes of priming the system or returning blood to the patient as described above. A simple optical detector with a light source and a photodetector can also be used to detect whether there is blood in the system.

In most renal diseases, the kidneys fail to produce erythropoietin, a hormone that stimulates red blood cell production. Most ESRD patients take an erythropoietin stimulation drug to help produce red blood cells. These drugs are dosed to maintain a pre-treatment serum hematocrit of 32%. During the course of the dialysis treatment, the hematocrit can change due to the removal of fluid from the blood. Hematocrit level changes over the course of the treatment are an indication of relative blood volume changes over treatment. Fluid removal by ultrafiltration removes fluid from the blood plasma; however, red blood cells are left in the circulatory system. Depending on the rate of vascular fluid refilling from the tissues, the hematocrit will increase or decrease. A flat hematocrit indicates that the patient is most likely fluid overloaded even at the end of therapy. A steep increase in the slope of the hematocrit during fluid removal may portend a hypovolemic event prior to initiating a hypotensive episode. A gradual increase in hematocrit during the course of treatment is most likely indicative of a well-dialyzed patient.

Hematocrit level is proportional to hemoglobin concentration. Therefore, any suitable sensor can be used to measure hemoglobin concentration, such as sensors used in pulse oximeters which measure adsorption of red and infrared light to determine concentration of oxygenated hemoglobin and deoxyhemoglobin, respectfully. The hematocrit/hemoglobin sensors, which may include the associated light source(s), can be placed in any suitable location. Placement of the hematocrit/hemoglobin sensor along the arterial line 610 of the extracorporeal circuit 140 will indicate the status of blood volume within the circulation of the patient. Placement of the hematocrit/hemoglobin sensor along the venous line 620 of the extracorporeal circuit 140 will indicate the extent of hemoconcentration occurring within the dialyzer 130. Measurement of hematocrit within the arterial line 610 can be used to calculate Qp as described above. Other optical based technologies that can determine the relative blood volume changes during the course of treatment can also be used to determine hydration status of the patient and whether the appropriate amount of fluid has been removed

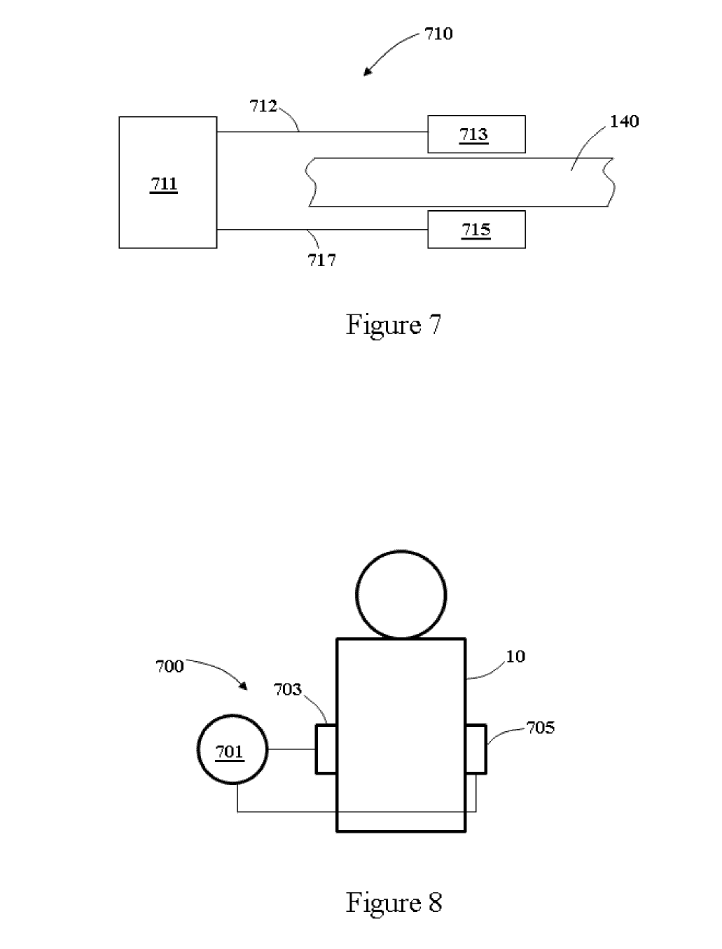

FIG. 7 shows a schematic for a hematocrit/hemoglobin/relative blood volume sensor. A light source 713 of appropriate wavelength (red or infrared) is positioned on one side of the tubing of extracorporeal circuit 140 such that the light passing through tubing hits detector 715. More light is absorbed (and less hits the detector 715) if a higher concentration of hemoglobin is present in the extracorporeal circuit 140. A lead 712 carries power and other electrical signals, if appropriate, to the light source 713 from the sensor device body 711, which may contain the power source and other control or detecting electronics. Lead 717 carries electrical signals from detector 715 to the components housed in sensor device body 711. Suitable hematocrit sensors are known, such as a CRIT-LINE monitor from HEMAMETRICS (see, HEMAMETRICS, CRIT-LINE hematocrit accuracy, Vol. 1, Techn Note No. 11 (Rev. D) Feb. 24, 2003).

In other embodiments, hemoconcentration can be detected and monitored by a relative blood volume monitor. The relative blood volume monitor can detect a change in the concentration of measured solutes, solid materials or group of solutes and solid materials in the blood that are too large to cross the dialysis membrane 135, which indicates a change in blood volume. The volume of blood typically is not measured by the relative blood volume monitor. That is, the relative blood volume monitor measures the change in water content of the blood over the course of treatment indirectly by measuring a solute or solid material and does not require an absolute quantization of any particular solute in the blood. The relative blood volume monitor determines the relative blood volume hydration status (RBVHS) of the subject by measuring the level of one or more blood solutes at a time close to the beginning of treatment, which can be assigned a value C₀. The level of the one or more blood solutes does not require an absolute quantification, rather the level of the one or more blood solutes can be reported as the magnitude of a signal generated by the relative blood volume monitor. The level of the one or more solutes is measured periodically at a second later time, which can be assigned a value Cₜ. The relative blood volume hydration status can then be determined by the formula RBVHS=C₀/Cₜ.

In certain embodiments, the relative blood volume monitor is a hematocrit sensor and the one or more solutes measured by the relative blood volume monitor are oxygenated or deoxygenated hemoglobin. In certain other embodiments, the relative blood volume monitor is a device that measures the velocity of ultrasonic sound waves in the blood. Ultrasonic sound waves are defined as sound waves having a frequency above 20,000 Hz. The velocity of ultrasonic sound waves in blood is an indication of the total protein concentration in the blood.

The relative blood volume hydration status can be used in the same manner as hematocrit, described above, to determine the effectiveness of ultrafiltration. It is important to note that when using relative blood volume the trend slope is inverse to the trend slope when using a hematocrit sensor, i.e. as hematocrit increases, relative blood volume decreases. A flat relative blood volume hydration status indicates that the patient is most likely fluid overloaded even at the end of therapy. A steep decrease in the slope of the relative blood volume hydration status during fluid removal can portend a hypovolemic event prior to initiating a hypotensive episode. A gradual decrease in relative blood volume hydration status during the course of treatment is most likely a well-dialyzed patient. In certain further embodiments, the relative blood volume hydration status determined by the relative blood volume monitor can be correlated to a fluid volume of the blood.

In the event that an unsafe level of hydration status is indicated by hematocrit level or by relative hydration status, a controller 801 associated with the system can stop the fluid removal and alert the patient. Controller 801 can also be programmed to remove fluid via a gradual slope in relative blood volume or hematocrit. Additionally, the controlled compliant nature of the dialysis circuit can be used to administer a bolus transfer of fluid to the patient. As described above, operation of the control pump 190 in the influx direction will cause a transfer of fluid volume from the control reservoir 191 to the extracorporeal circuit 140. The system can be preprogrammed to transfer a certain bolus volume to the patient upon detection of an unsafe trend in hematocrit or relative blood volume hydration status.

In certain embodiments, the control reservoir 191 is empty at the beginning of a treatment session wherein volume enters the control reservoir during treatment including ultrafiltration. As such, a bolus infusion in response to trend in hematocrit or relative blood volume hydration status is a return of fluid volume removed from the patient during treatment back to the patient. Any volume returned to the patient from the control reservoir 191 is cleaned by the sorbent cartridge 102 prior to introduction to the extracorporeal circuit 140. However, in other embodiments the control reservoir 191 can contain a volume of fluid at the beginning of treatment that can be used for a net infusion of fluid into the patient during the course of treatment.

Hypovolemia can further be guarded against by simultaneously monitoring body fluid level of the patient undergoing hemodialysis treatment. The amount of fluid stored in body tissues outside the blood is proportional to the impedance that can be measured from the patient's body. As depicted in FIG. 8, impedance can be monitored between two electrodes 703 and 705 that are attached to the torso 10 of a human patient. The electrodes 703 and 705 are operably coupled to control and processing electronics 701 via leads. The electronics 701 are configured to generate a voltage differential between the electrodes 703 and 705, and current can be measured and impedance calculated. The measurement can be done in either DC or AC mode. Impedance or phase angle can be correlated to tissue fluid volume. Suitable external impedance monitors 700 and components that can be used in accordance with the teachings described herein are known. In certain other embodiments, electrodes 703 and 705 can be implanted within the patient.

One example of a well studied system that can be used or modified for use herein is Medtronic, Inc.'s OptiVol® fluid status monitoring system. Such a system, or other similar systems, have well-documented procedures for determining acceptable ranges of tissue impedance and thus fluid volume. See, e.g., Siegenthalar, et al. Journal of Clinical Monitoring and Computing (2010): 24:449-451, and Wang, Am. J. Cardiology, 99(Suppl):3G-1-G, May 21, 2007. Alternatively or in addition, tissue impedance can be monitored for a suitable period of time to establish as suitable baseline, and patient markers can be used to instruct whether the patient is fluid overloaded or under-loaded. The data acquired by impedance sensor and input data regarding fluid status of the patient at the time the sensor data is acquired may be used to establish suitable ranges for impedance values.

One or more controllers 801 associated with the hemodialysis system can monitor the hematocrit/relative blood volume hydration status and impedance/body fluid level of the patient undergoing hemodialysis treatment. A typical hematocrit level for a dialysis patient is 32%. Prior to a treatment session, the fluid volume of blood of a kidney disease patient can be elevated, thus hematocrit levels can be lower than desired. The one or more controllers 801 monitoring hematocrit levels can adjust the rate of fluid removal or end ultrafiltration treatment when hematocrit level reaches the desired, predetermined range.

Fluid within a person's body is capable of moving from the body tissue to the blood and vice versa. Proper fluid levels in a patient can be described in terms of a ratio of tissue fluid to blood volume, as measured by hematocrit level. Hematocrit level of body fluid level can be monitored independently as described above. In general, blood is about 7% of body weight and total tissue fluid is about 60% of the body weight (including blood, extracellular and intracellular fluid). Typical tissue fluid to blood fluid volume ratio of a healthy individual is in the range from about 6:1 to about 9:1. A measured ratio above this range indicates that blood is being withdrawn too quickly to allow for adequate equilibration of fluid between the blood and tissues of the patient. Fluid removal can be modified, stopped, or a fluid bolus administered as appropriate and preprogrammed into the one or more controllers 801 of the hemodialysis system.

### Detection of Needle or Catheter Disconnection

It is well established in the art that pressure is not always a reliable means to detect separations of the venous blood return from the access of the patient. If this event occurs there is the risk of a life threatening blood loss and possible exsanguination. A conductive mat or holder can be used to detect blood leaks to the controller. The controller can then take the appropriate means to protect the patient by stopping the blood pump and alerting the patient. Other means to detect needle or catheter disconnections can be incorporated into the system such as monitoring of the impedance through the two needles or using pressure pulses.

### System Control

As described above, the systems described herein have several dynamic components including pumps and valves as well as detectors that determine the state of the system. As applied throughout this disclosure, operation of the system under the control of controller can refer to a single controller or multiple controllers having separate or overlapping function. A controller refers to a device having a programmable microprocessor and associated memory.

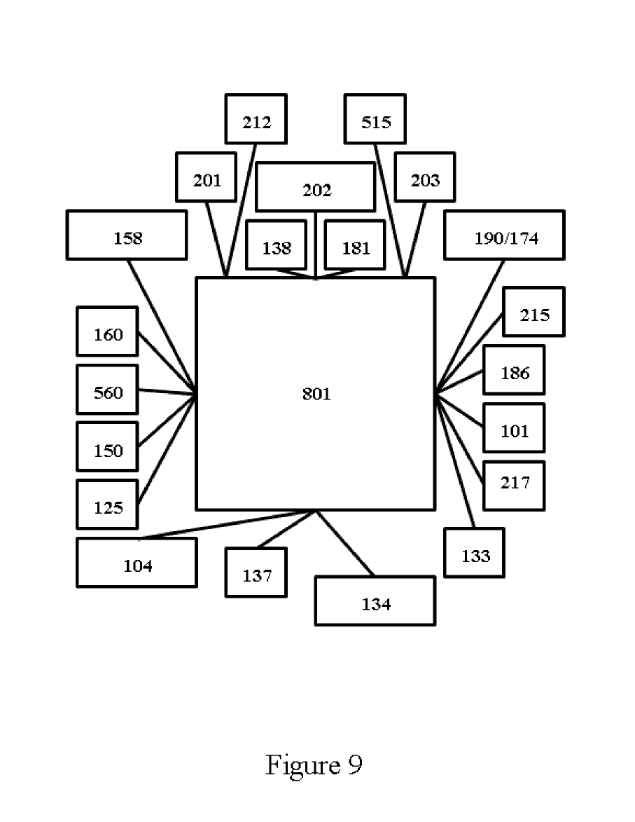

FIG. 9 shows one or more controllers 801 capable of sending and receiving data or instructions from several system components. The one or more controllers 801 can be more than one microprocessor unit. Specifically, the one or more controllers 801 are capable of controlling the pump rate and pumping direction of the blood pump 125, the dialysate pump 138 and the control pump 190 along with the operating of valve 150, valve 251, valve 158, valve 515, and valve 212. The operation of anticoagulant pump 186, bicarbonate pump 505 and infusate pump 181 is further under control of the one or more controllers 801. In two controller systems one controller may be used to control the process and the other controller may be used to monitor the system and protect if the control is not correct. Alternatively in one-controller systems, the processes that control or protect may be separate processes within the same controller.

The one or more controllers 801 also receives data from the various meters and detectors incorporated in the system including pressure meters 133, 134, and 137, detectors 201, 202, and 203, conductivity detectors 101, 104 and 160 and blood leak detector 560. The one or more controllers 801 are capable of stopping or modifying operation of the system to protect the patient from an unsafe pressure reading indicating a malfunction or the presence of air in the extracorporeal circuit 140, an unsafe conductivity level or detection of a blood leak in the dialyzer 130, as detected by blood leak detector 560. The one or more controllers are capable of stopping any of the pumps of the systems or operating valve 150 to bypass the dialyzer 130. Further, the one or more controllers 801 can modify or stop the operation of the system based upon the conductivity readings from the conductivity meters 101, 104 and 160 as well as calculating an amount of urea absorption by the sorbent cartridge 102 and/or sodium entering or leaving the system through control pump 190.

In certain embodiments, the one or more controllers 801 are located remote from the dialysis and extracorporeal circuits. One of the controllers 801 can be a device that can send and receive data and instructions through a wired or wireless connection with the portable dialysis system. Certain controller functions, for example, can be performed by an application that runs on a multipurpose computing device such as a cell phone, tablet, PC or PDA. In certain embodiments, a controller 801 that is remote to the portable dialysis system is capable of operating through a wired connection to the portable dialysis system to enable operation in hospital environments or airplanes where the use of wireless technology is restricted.

By locating one or more of the controllers 801 remote from the portable dialysis system, the majority of processing power does not have to be carried by the patient thereby lowering the weight of the device. Devices and methods for controlling a device through wireless technology are known in the art. The wireless signals can employ signal confirmation, digital encoding algorithms, checksums and other verifications to minimize the effects of interference and to allow similar systems to operate in the same area. The system can have a safety feature to stop the device if the wireless control signal is interrupted or compromised.

It will be apparent to one skilled in the art that various combinations and/or modifications and variations can be made to the portable dialysis system depending upon the specific needs for operation. Moreover, features illustrated or described as being part of one embodiment may be used on another embodiment to yield a still further embodiment.

We claim:

1. A cartridge, comprising: a polystyrene sulfonate resin substantially saturated with calcium ions capable of removing one or more of sodium ions, carbonate ions, sulfate ions and phosphates from a fluid passing through the cartridge.

2. The cartridge of claim 1, wherein the polystyrene sulfonate resin predominantly contains the following structure: 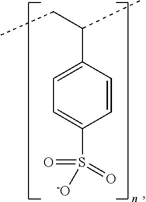
where n is an integer from about 5 to about 200.

3. The cartridge of claim 1, wherein the polystyrene sulfonate resin saturated with calcium ions ((RSO₃⁻)₂Ca²⁺) precipitates carbonate and absorbs sodium ions through the following reactions:
(RSO₃⁻)₂Ca²⁺+Na⁺ [symbol] 2RSO₃⁻Na⁺+Ca²⁺ (1)
Ca²⁺(aq)+CO₃²⁻(aq)→CaCO₃ (2).

4. The cartridge of claim 1, wherein the polystyrene sulfonate resin saturated with calcium ions ((RSO₃⁻)₂Ca²⁺) precipitates sulfate and absorbs sodium ions through the following reactions:
(RSO₃⁻)₂Ca²⁺+Na⁺ [symbol] 2RSO₃⁻Na⁺+Ca²⁺ (1)
Ca²⁺(aq)+SO₄²⁻(aq)→CaSO₄(s) (3).

5. The cartridge of claim 1, wherein the polystyrene sulfonate resin saturated with calcium ions ((RSO₃⁻)₂Ca²⁺) precipitates HₓPO₄^{3-x} species and absorbs sodium ions through the following reactions:
(RSO₃⁻)₂Ca²⁺+Na⁺ [symbol] 2RSO₃⁻Na⁺+Ca²⁺ (1)
Ca²⁺(aq)+HₓPO₄^{x-3}(aq)→Ca₃(PO₄)₂(s)+xH⁺ (4),
where x is an integer from 0 to 2.

6. A method for performing kidney replacement therapy, comprising:
conveying a dialysate through a dialysis circuit via a pump such that the pump conveys the dialysate from a sorbent cartridge, to a dialyzer and back to the sorbent cartridge, wherein at least one impurity or waste species diffuses into the dialysate via the dialyzer and the sorbent cartridge removes the waste species from the dialysate and releases sodium ions into the dialysate; and
intermittently conveying the dialysate through a polystyrene sulfonate resin cartridge, wherein the polystyrene sulfonate resin cartridge contains a polystyrene sulfonate resin substantially saturated with calcium ions, and the polystyrene sulfonate resin cartridge removes one or more selected from sodium ions, carbonate ions, sulfate ions and phosphates from the dialysate.

7. The method of claim 6, wherein the dialysate is intermittently conveyed through the polystyrene sulfonate resin as the dialysate is being conveyed from the dialyzer to the sorbent cartridge or from the sorbent cartridge to the dialyzer

8. The method of claim 6, wherein the polystyrene sulfonate resin predominantly contains the following structure: 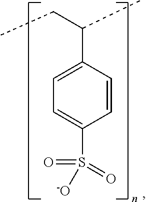
where n is an integer from about 5 to about 200.

9. The method of claim 6, wherein the polystyrene sulfonate resin substantially saturated with calcium ions ((RSO₃⁻)₂Ca²⁺) precipitates carbonate and absorbs sodium ions through the following reactions:
(RSO₃⁻)₂Ca²⁺+Na⁺ [symbol] 2RSO₃⁻Na⁺+Ca²⁺ (1)
Ca²⁺(aq)+CO₃²⁻(aq)→CaCO₃ (2).

10. The method of claim 6, further comprising:
monitoring the conductivity of the dialysate at an inlet end of the sorbent cartridge via a first conductivity meter;
monitoring the conductivity of the dialysate at an outlet end of the sorbent cartridge via a second conductivity meter; and
calculating an amount of urea absorbed by the sorbent cartridge based at least in part upon the conductivity measured at the inlet end of the sorbent cartridge and at the outlet end of the sorbent cartridge.

11. The method of claim 10, wherein the amount of urea absorbed by the sorbent cartridge is calculated by:
calculating a starting conductivity by subtracting a conductivity attributed to Ca²⁺, Mg²⁺, and K⁺ ions in the dialysate from a conductivity measured at the inlet of the sorbent cartridge;
calculating a corrected outlet conductivity by subtracting an increase in conductivity attributed to an exchange of Ca²⁺, Mg²⁺, and K⁺ ions for Na⁺ ions by the sorbent cartridge from a conductivity measured at the outlet of the sorbent cartridge; and
calculating a conductivity increase from the exchange of NH₄⁺ for Na⁺ ions by the sorbent cartridge by subtracting the starting conductivity from the corrected outlet conductivity.

12. The method of claim 6, wherein the dialysis circuit is a controlled compliance circuit.

13. The method of claim 6, further comprising controlling a ratio of dialysis flow passing through the polystyrene sulfonate resin cartridge and dialysis flow passing through only the sorbent cartridge, wherein the dialysis flow passing through the polystyrene sulfate resin cartridge is represented by R₁, the dialysate flow bypassing the polystyrene sulfate resin cartridge is represented by R₀ and the ratio is represented by R₁/R₀, wherein the ratio R₁/R₀ is controlled based upon the conductivity of the dialysate.

14. The method of claim 13, further comprising:
monitoring the conductivity of the dialysate at an inlet end of the sorbent cartridge via a first conductivity meter;
monitoring the conductivity of the dialysate at an outlet end of the sorbent cartridge via a second conductivity meter; and
calculating an amount of urea absorbed by the sorbent cartridge based at least in part upon the conductivity measured at the inlet end of the sorbent cartridge and at the outlet end of the sorbent cartridge, wherein the ratio R₁/R₀ is controlled based upon an amount of urea absorbed by the sorbent cartridge.

15. The method of claim 13, further comprising monitoring a concentration of calcium ions of the dialysate using a calcium sensing electrode, wherein the ratio R₁/R₀ is controlled based upon the concentration of calcium ions in the dialysate.

16. A system for performing kidney replacement, comprising:
an extracorporeal circuit and a dialysis circuit, the dialysis circuit having a sorbent cartridge for removing at least one waste species from a dialysate, one or more conduits for carrying dialysate between the sorbent cartridge and a dialyzer, and a dialysate pump for conveying dialysate from the sorbent cartridge, to the dialyzer and back to the sorbent cartridge; and
a polystyrene sulfonate resin cartridge and one or more conduits for carrying dialysate from the dialysis circuit through the polystyrene sulfonate resin cartridge, and
one or more controllers for controlling a flow of the dialysate through the sorbent cartridge and through the polystyrene sulfonate resin cartridge,
wherein the polystyrene sulfonate resin cartridge contains a polystyrene sulfonate resin substantially saturated with calcium ions and the polystyrene sulfonate resin cartridge removes one or more selected from the group consisting of sodium ions, carbonate ions, sulfate ions and phosphates from the dialysate.

17. The system of claim 16, further comprising:
a first conductivity meter for measuring the conductivity of the dialysate at a dialysate inlet end of the sorbent cartridge; and
a second conductivity meter for measuring the conductivity of the dialysate at a dialysate outlet end of the sorbent cartridge, wherein
the one or more controllers for compare the conductivity measured by the first conductivity meter and the second conductivity meter to calculate the amount of urea absorbed by the sorbent cartridge.

18. The system of claim 16, wherein the one or more controllers control a ratio of dialysate flow flowing through the polystyrene sulfonate resin cartridge and dialysate flow passing through only the sorbent cartridge, wherein the dialysate flow passing through the polystyrene sulfonate resin cartridge is represented by R₁, dialysate flow bypassing the polystyrene sulfonate resin cartridge is represented by R₀ and the ratio is represented by R₁/R₀, wherein the ratio R₁/R₀ is controlled based upon the conductivity of the dialysate.

19. The system of claim 18, wherein the controller monitors the conductivity of the dialysate at an inlet end of the sorbent cartridge, the conductivity of the dialysate at an outlet end of the sorbent cartridge; and calculates an amount of urea absorbed by the sorbent cartridge based at least in part upon the conductivity measured at the inlet end of the sorbent cartridge and at the outlet end of the sorbent cartridge, wherein the ratio R₁/R₀ is controlled based upon an amount of urea absorbed by the sorbent cartridge.

20. The system of claim 18, wherein the controller monitors a concentration of calcium ions of the dialysate using a calcium sensing electrode, wherein the ratio R₁/R₀ is controlled based upon the concentration of calcium ions in the dialysate.
